SEQUENCE LISTING (27 sequences)

SEQ ID NO: 1 - protein, 5 aa, Mus musculus
  /note=“Description amino acid sequence of CDR1”
SYAMS

SEQ ID NO: 2 - protein, 16 aa, Mus musculus
  /note=“Description amino acid sequence of CDR2”
SIGSGGGTYYPDSVKD

SEQ ID NO: 3 - protein, 13 aa, Mus musculus
  /note=“Description amino acid sequence of CDR3”
AYYRNHRGSVMDY

SEQ ID NO: 4 - protein, 15 aa, Mus musculus
  /note=“Description amino acid sequence of CDR1′”
KASQTVDYDGDSYMN

SEQ ID NO: 5 - protein, 7 aa, Mus musculus
  /note=“Description amino acid sequence of CDR2′”
SASNLES

SEQ ID NO: 6 - protein, 9 aa, Mus musculus
  /note=“Description amino acid sequence of CDR3′”
QQSTEDPPT

SEQ ID NO: 7 - DNA, 363 bp, Mus musculus
  /note=“Description nucleic acid sequence encoding VH”
gaagtgaagctggtggagtctgggggagtcttagtgaagcctggagggtccctgaaactctcctgtgcagcctctggattcactttcagtagctatgccatgtcttgggttcgccagactccagaaaagaggctggagtgggtcgcatctattggtagtggtggtggaacttactatccagacagtgtgaagggccgattcaccatctccagaaataatgccaggaacatcctgtacctgcaaatgagcagtctgaggtctgaggacacggccatgtattactgtgcaagagcctactataggaaccaccgagggtctgttatggactactggggtcaaggaacctcagtcaccgtctcctca

SEQ ID NO: 8 - protein, 121 aa, Mus musculus
  /note=“Description amino acid sequence of VH”
EVKLVESGGVLVKPGGSLKLSCAASGFTFSSYAMSWVRQTPEKRLEWVASIGSGGGTYYPDSVKGRFTISRNNARNILYLQMSSLRSEDTAMYYCARAYYRNHRGSVMDYWGQGTSVTVSS

SEQ ID NO: 9 - DNA, 333 bp, Mus musculus
  /note=“Description nucleic acid sequence encoding VL”
gacattgtgctgacccaatctccagcttctttggctgtgtctctagggcagagggccaccatctcctgcaaggccagccaaactgttgattatgatggtgatagttatatgaactggtaccaacagaaaccaggacagccacccaaagtcctcatctattctgcatccaatctggaatctgggatcccagccaggtttagtggcagtgggtctgggacagacttcaccctcaacatccatcctgtggaggaggaggatgctgcaacctattactgtcagcaaagtactgaggatcctccgacgttcggtggaggcaccaagctggaaatcaaa

SEQ ID NO: 10 - protein, 111 aa, Mus musculus
  /note=“Description amino acid sequence of VL”
DIVLTQSPASLAVSLGQRATISCKASQTVDYDGDSYMNWYQQKPGQPPKVLIYSASNLESGIPARFSGSGSGTDFTLNIHPVEEEDAATYYCQQSTEDPPTFGGGTKLEIK

SEQ ID NO: 11 - DNA, 45 bp, Mus musculus
  /note=“Description nucleic acid sequence encoding CDR1′”
aaggccagccaaactgttgattatgatggtgatagttatatgaac

SEQ ID NO: 12 - DNA, 21 bp, Mus musculus
  /note=“Description nucleic acid sequence encoding CDR2′”
tctgcatccaatctggaatct

SEQ ID NO: 13 - DNA, 27 bp, Mus musculus
  /note=“Description nucleic acid sequence encoding CDR3′”
cagcaaagtactgaggatcctccgacg

SEQ ID NO: 14 - DNA, 15 bp, Mus musculus
  /note=“Description nucleic acid sequence encoding CDR1”
agctatgccatgtct

SEQ ID NO: 15 - DNA, 48 bp, Mus musculus
  /note=“Description nucleic acid sequence encoding CDR2”
tctattggtagtggtggtggaacttactatccagacagtgtgaagggc

SEQ ID NO: 16 - DNA, 39 bp, Mus musculus
  /note=“Description nucleic acid sequence encoding CDR3”
gcctactataggaaccaccgagggtctgttatggactac

SEQ ID NO: 17 - DNA, 843 bp, Bos taurus
  /note=“Description cDNA sequence encoding bovine CD40”
atggttcgtttgccactgcagtgtctcttctggggcttctttctgaccgccgtccactcagaaccagccactgcttgtggagagaagcaatacccagtgaacagtctttgctgtgatttgtgcccgccgggacagaaactggtgaacgactgcacagaggtcagcaaaacagaatgccagtcctgcggtaaaggcgaattcttgtccacctggaacagagagaaatactgtcacgagcacagatactgcaaccccaacctagggctccggatccagagcgagggtaccttgaatacagacaccatttgtgtatgtgtcgaaggccaacactgtaccagtcacacctgcgaaagttgcacgccccacagcttgtgtctccctggcttcggggtcaagcagatcgctacagggcttttggataccgtctgtgaaccctgcccgctcggcttcttctccaacgtgtcatctgcttttgaaaagtgtcaccgttggacaagctgcgagagaaaaggcctggtggaacaacacgtggggacgaacaagacagatgttgtctgcggtttccagagtcggatgaggaccctggtggtgatccccgtcacgatgggagtcttgtttgctgtcctgttggtatctgcctgtatcaggaacataaccaagaagcggcaggctaaggccctgcaccctatggctgaaaggcaggatcccgtggagacgattgatccggaggattttcccggcccccacccgcctcctccggtgcaagagaccttatgctggtgtcagccggtcgcccaggaggacggcaaagagagccgcatctccgtgcaggagcgagaatga

SEQ ID NO: 18 - protein, 280 aa, Bos taurus
  /note=“Description bovine CD40 amino acid sequence”
MVRLPLQCLFWGFFLTAVHSEPATACGEKQYPVNSLCCDLCPPGQKLVNDCTEVSKTECQSCGKGEFLSTWNREKYCHEHRYCNPNLGLRIQSEGTLNTDTICVCVEGQHCTSHTCESCTPHSLCLPGFGVKQIATGLLDTVCEPCPLGFFSNVSSAFEKCHRWTSCERKGLVEQHVGTNKTDVVCGFQSRMRTLVVIPVTMGVLFAVLLVSACIRNITKKRQAKALHPMAERQDPVETIDPEDFPGPHPPPPVQETLCWCQPVAQEDGKESRISVQERE

SEQ ID NO: 19 - DNA, 21 bp, Artificial Sequence
  Source: /note=“Description of Artificial Sequence Primer nucleic acid sequence F-CD40”
atggttcgtttgccactgcag

SEQ ID NO: 20 - DNA, 20 bp, Artificial Sequence
  Source: /note=“Description of Artificial Sequence Primer nucleic acid R-CD40”
tcattctcgctcctgcacgg

SEQ ID NO: 21 - DNA, 24 bp, Artificial Sequence
  Source: /note=“Description of Artificial Sequence Nucleic acid sequence coding for FLAG epitope”
gactacaaggacgacgatgacaag

SEQ ID NO: 22 - protein, 8 aa, Artificial Sequence
  Source: /note=“Description of Artificial Sequence Peptide amino acid sequence of FLAG epitope”
DYKDDDDK

SEQ ID NO: 23 - DNA, 70 bp, Artificial Sequence
  Source: /note=“Description of Artificial Sequence Primer nucleotide sequence F1”
tttctgaccgccgtccactcagactacaaggacgacgatgacaaggaaccagccactgcttgtggagaga

SEQ ID NO: 24 - DNA, 72 bp, Artificial Sequence
  Source: /note=“Description of Artificial Sequence Primer nucleotide sequence F2”
atcgccaccatggttcgtttgccactgcagtgtctcttctggggcttctttctgaccgccgtccactcagac

SEQ ID NO: 25 - DNA, 28 bp, Artificial Sequence
  Source: /note=“Description of Artificial Sequence Primer nucleotide sequence R”
ataatcattctcgctcctgcacggagat

SEQ ID NO: 26 - protein, 261 aa, Homo Sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 27 - protein, 261 aa, Bos taurus
MIETYSQPSPRSVATGPPVSMKIFMYLLTVFLITQMIGSALFAVYLHRRLDKIEDERNLHEDFVFMKTIQRCNKGEGSLSLLNCEEIRSRFEDLVKDIMQNKEVKKKEKNFEMHKGDQEPQIAAHVISEASSKTTSVLQWAPKGYYTLSNNLVTLENGKQLAVKRQGFYYIYTQVTFCSNRETLSQAPFIASLCLKSPSGSERILLRAANTHSSSKPCGQQSIHLGGVFELQSGASVFVNVTDPSQVSHGTGFTSFGLLKL

(19) United States
(12) Patent Application Publication
(10) Pub. No.: US 2014/0050732 A1
(43) Pub. Date: Feb. 20, 2014

(54) COMBINATION FOR USE IN THE TREATMENT AND/OR PREVENTION OF MASTITIS

(71) Applicant: UNIVERSITE DE LIEGE, Angleur (BE)

(72) Inventors: Fabrice Bureau, Ransart (BE); Hughes Wallemacq, Fexhe-Slins (BE); Philippe Boutet, Louveigne (BE); Pierre Lekeux, Braine-I'Alleud (BE); Laurence Fievez, Oteppe (BE); Julien Pujol, Douvaine (BE)

(21) Appl. No.: 13/841,591

(22) Filed: Mar. 15, 2013

Related U.S. Application Data
(63) Continuation-in-part of application No. PCT/EP2011/065151, filed on Sep. 1, 2011.

(30) Foreign Application Priority Data
Sep. 29, 2010 (EP) 10182537.0

(51) Int. Cl. A61K 39/39; A61K 39/085; C07K 14/705; A61K 39/02; A61K 39/108; C07K 16/28; A61K 39/395; A61K 39/09

(52) U.S. Cl. CPC A61K 39/39; A61K 39/3955; A61K 39/085; A61K 39/092; A61K 39/0208; A61K 39/0258; C07K 16/2878; C07K 14/70575
USPC 424/139.1; 424/235.1; 424/243.1; 424/244.1; 424/234.1; 424/257.1; 536/23.53; 435/320.1; 536/23.5; 435/331; 435/252.33; 435/252.31; 435/254.11; 435/325; 435/254.2; 435/254.21; 435/348; 435/369; 435/354; 435/365; 435/350; 435/357; 435/372.3

(57) ABSTRACT

The present invention relates a combination for use in the treatment and/or prevention of mastitis containing i) an agonistic anti-CD40 monoclonal antibody or a CD40 ligand or a vector coding for the anti-CD40 antibody or a vector coding for the CD40L; and ii) inactivated or attenuated bacteria selected from the group consisting of Staphylococcus, Streptococcus, Listeria or Escherichia.

This application is a continuation-in-part (CIP) of PCT/EP2011/065151, filed Sep. 1, 2011, which claims the priority of European Patent Application No. 10182537.0, filed Sep. 29, 2010, the disclosures of which are incorporated herein by reference.

### FIELD OF THE INVENTION

The present invention relates to combinations or compositions useful in the treatment or prevention of mastitis. Specifically, the combinations or compositions contains an agonistic anti-CD40 monoclonal antibody or CD40 ligand (CD40L) or a vector coding for the anti-CD40 antibody or a vector coding for the CD40L, and inactivated or attenuated bacteria selected from the group consisting of Staphylococcus, Streptococcus, Listeria or Escherichia.

### BACKGROUND OF THE INVENTION

Mastitis is the most costly disease affecting dairy cattle worldwide. Staphylococcus (S.) aureus, a common gram-positive bacterium, is the most prevalent infectious agent that affects the bovine udder. After entering the mammary gland, S. aureus multiplies rapidly and causes tissue damages, leading to reduction in both the quantity and quality of the milk. Staphyloccocal mastitis remains difficult to control efficiently [2]. This may be related to the ability of S. aureus to invade and survive within host phagocytes and mammary epithelial cells [3, 4]. Indeed, intracellular invasion provides protection from the humoral immune response and several classes of antibiotics.

Numerous vaccine strategies have been developed in order to increase herd resistance to S. aureus mastitis and to reduce the clinical and economic consequences of this disease [2]. While some formulations have shown promise in ameliorating the disease, few, if any, of the S. aureus vaccines developed have adequately prevented new infections [2]. Several reasons could account for the lack of efficient S. aureus vaccines. First, although a number of virulence factors have been suggested as potential antigens for single-component vaccines, experimental trials have demonstrated that induction of immunity to single factors is not sufficient to confer robust protection against S. aureus [2]. Second, although killed and live attenuated vaccines have the advantage that they represent a greater pool of antigens and are considered an attractive alternative approach, they suffer from low immunogenicity and require adequate adjuvants [2]. Third, the major challenge in the control of staphylococcal mastitis is to efficiently target intracellular bacteria. However, commonly used adjuvants, such as alum and incomplete Freund's adjuvant, predominantly enhance humoral responses and there is currently no available vaccine able to elicit strong CD8 cytotoxic T lymphocyte (CTL) responses to S. aureus.

Consequently, it is recognized in the art that there is a need to find a strategy to overcome the above-mentioned lack of efficient vaccines directed against intracellular pathogens in cattle. Accordingly, the problem underlying the present invention is the provision of means and methods for the treatment as well as the potential prevention of infectious diseases in cattle.

### SUMMARY OF THE INVENTION

This technical problem is solved by the embodiments provided herein and as characterized in the claims. Specifically and in accordance with the present invention, a solution to this technical problem is achieved by providing a combination of i) an agonistic anti-CD40 monoclonal antibody or a CD40 ligand (CD40L) (or fragments thereof) or a vector coding for the anti-CD40 antibody or a vector coding for the CD40L; and ii) inactivated or attenuated bacteria selected from the group consisting of Staphylococcus, Streptococcus, Listeria or Escherichia for use in the treatment and/or prevention of mastitis. Preferably, the CD40L (or fragments thereof) is in a fusion protein with glutathione S-transferase (GST).

Preferably, the agonistic anti-CD40 monoclonal antibody as provided comprises a) an immunoglobulin heavy chain variable domain (VH) which comprises the hypervariable regions CDR1, CDR2 and CDR3, said CDR1 having the amino acid sequence SEQ ID NO:1: SYAMS, said CDR2 having the amino acid sequence SEQ ID NO:2: SIGSGGGTYYPDSVKD, and said CDR3 having the amino acid sequence SEQ ID NO:3: AYYRNHRGSVMDY; and b) an immunoglobulin light chain variable domain (VL) which comprises the hypervariable regions CDR1′, CDR2′ and CDR3′, said CDR1′ having the amino acid sequence SEQ ID NO:4: KASQTVDYDGDSYMN, said CDR2′ having the amino acid sequence SEQ ID NO:5: SASNLES, and said CDR3′ having the amino acid sequence SEQ ID NO:6: QQSTEDPPT; for use in the treatment and/or prevention of mastitis; or variants thereof capable of inducing CD40 receptor aggregation. Preferably, the CD40L as provided contains the amino acids 47 to 261 or 46 to 261 of SEQ ID NO: 26 or SEQ ID NO: 27, or fragments thereof. Furthermore, the present invention relates to a nucleic acid molecule encoding the agonistic anti-CD40 monoclonal antibody or CD40L, a vector comprising said nucleic acid, and a host comprising said vector. Finally, the present invention relates to a vaccine comprising the combination of an agonistic anti-CD40 monoclonal antibody or a CD40 ligand (CD40L) or a vector coding for the anti-CD40 antibody or a vector coding for the CD40L, and inactivated or attenuated bacteria selected from the group consisting of Staphylococcus, Streptococcus, Listeria or Escherichia.

### BRIEF DESCRIPTION OF THE DRAWINGS

FIG. 1: Immunization with OVA in conjunction with αCD40 induces IFN-γ-dependent immune responses in the mammary gland. Naive C57BL/6 mice were injected s.c. between the L4 and R4 abdominal mammary glands with 10 μg OVA and immediately afterward were given 25 μg αCD40 i.p. (OVA/αCD40 group). Control mice received either OVA alone (10 μg; s.c.), αCD40 alone (25 μg; i.p.), or PBS (100 μl s.c. and i.p.). Five days after immunization, MLN cells were isolated and restimulated in vitro for 3 days with 50 μg/ml OVA. (A) The proliferation was measured as ³H-thymidine incorporation during the last 16 hr of culture. (B) Culture supernatants were assayed for IFN-γ and IL-4 by ELISA. (A and B)* significantly different from the other values (P<0.05).

FIG. 2: Proliferation of OVA-specific CD4⁺ and CD8⁺ T cells in the MLNs of OVA/αCD40 mice. Naive C57BL/6 were injected i.v. with 10⁶ CFSE-labelled OTI or OTII T cells (day −1). Twenty-four hr later (day 0), mice were injected with either PBS, OVA, αCD40, or OVA/αCD40. On day 3, MLNs were collected and proliferation of CFSE-labelled OVA-specific T cells was measured by flow cytometry.

FIG. 3: OVA/αCD40 immunization induces IFN-γ-secreting CD8⁺ T cells in the MLNs. Naive C57BL/6 mice were injected with either PBS (100 μl s.c. and i.p.), OVA alone (10 μg; s.c.), αCD40 alone (25 μg; i.p.), or OVA/αCD40. Five days after immunization, MLN cells were isolated and placed in culture. MLN cells were then treated with PMA (10 ng/ml) and ionomycin (250 ng/ml) for 6 hours and incubated with 1 μg/ml Brefeldin A for the last 4 hours. Cells were stained for either CD4 or CD8, fixed and permeabilized, and finally stained for intracellular IFN-γ.

FIG. 4: OVA/αCD40 immunization enhances the development of efficient cytotoxic CD8⁺ T cells. Naive C57BL/6 mice were injected with either PBS, OVA, αCD40, or OVA/αCD40. Five days later, the mice received, by the i.v. route, a mixture of OT-I peptide-pulsed CFSE^{high} and unpulsed CFSE^{lo} splenocytes (the two cell populations were mixed at a 1:1 ratio and 5×10⁷ cells were injected). MLNs were isolated 16 h later and single cell suspensions were analyzed by flow cytometry for quantification of CFSE-labelled cells.

FIG. 5: HKSA/αCD40 immunization induces specific IFN-γ-secreting CD8⁺ T cells MLNs. Naive C57BL/6 mice were injected s.c. between the L4 and R4 abdominal mammary glands with 5×10⁸ HKSA and immediately afterward were given 25 μg αCD40 i.p. (HKSA/αCD40 group). Control mice received either HKSA alone (5×10⁸; s.c.), αCD40 alone (25 μg; i.p.), or PBS (100 μl s.c. and i.p.). (A) Five days after immunization, MLN cells were isolated and restimulated in vitro for 3 days with 1×10⁵ HKSA. The proliferation was measured as ³H-thymidine incorporation during the last 16 hr of culture (upper panel). Culture supernatants were assayed for IFN-γ and IL-4 by ELISA (middle and lower panels). * significantly different from the other values (P<0.05). (B) Alternatively, MLN cells were stimulated in vitro with 1×10⁵ HKSA/ml for 20 hours and incubated with Brefeldin A for the last 4 hours. Cells were then stained for either CD4 or CD8, fixed and permeabilized, and finally stained for intracellular IFN-γ. Percentages of positive cells are given in the gates.

FIG. 6: HKSA/αCD40 vaccination requires CD8⁺ T cell induction to protect mice against staphylococcal mastitis. BALB/c lactating mice were immunized with HKSA (5×10⁸ CFU; s.c.) combined with αCD40 (25 μg; i.p.) 7 days after birth of the offspring. Control mice were administered with either PBS, HKSA alone, or αCD40 alone. In order to evaluate the contribution of CD8⁺ T cells to the effects observed, some HKSA/αCD40 mice were treated i.p. 3 days before and at the time of infection with 300 μg depleting anti-CD8 mAbs. Seven days after immunization, 10² CFU of S. aureus were injected in the lactiferous duct of both the L4 and R4 abdominal mammary glands. Twelve hours later, mammary glands were harvested, dissected and homogenized in PBS. Homogenates were serially diluted and plated on mannitol salt agar for CFU determination. Raw bacterial CFU counts were transformed in base-10 logarithm and data represented by medians. *, significantly different from the other values (Mann-Whitney U-test, P<0.01).

FIG. 7: Anti-mouse and anti-human CD40 antibodies are unable to induce IL-12 secretion by bovine DCs. Bovine DCs were generated by culturing peripheral blood CD14⁺ cells with bovine GM-CSF and IL-4. After five days of culture, DCs were stimulated for 24 hours with 1 μg/ml anti-mouse (clone 1C10) or anti-human (clone B-B20) CD40 antibodies. Cell culture supernatants were then assayed for the presence of bovine IL-12 by ELISA. Isotype control antibodies (Ctrl Abs) were used as negative controls. LPS was used as positive control of bovine DC stimulation.

FIG. 8: Validation of the anti-bovine CD40 antibodies. (A) Whole-cell extracts were prepared from bovine PBMCs or from COS-7 cells transfected with either the pcDNA3.1/CD40 vector (CD40) or the empty pcDNA3.1 vector (Empty). Lysates were then analyzed by immunoblotting for CD40 expression using polyclonal sera as primary antibodies. (B) COS-7 cells transfected with either the pcDNA3.1/CD40 vector (COS-7:CD40) or the empty pcDNA3.1 vector (COS-7/Empty) were stained for CD40 using the E1 monoclonal antibody as a primary antibody and a FITC-conjugated goat anti-mouse IgG as a secondary antibody. Cells were then analyzed for FITC fluorescence by flow cytometry. As a control, bovine CD40-transfected COS-7 cells were also stained using an isotype IgG2b control instead of the E1 monoclonal antibody. (C) Whole-cell extracts from bovine PBMCs were analyzed by immunoblotting for CD40 expression using the E1 monoclonal antibody.

FIG. 9: The E1 monoclonal anti-CD40 antibody induces IL-12 production by bovine DCs. Monocyte-derived bovine DCs at day 5 of culture were stimulated with 1 μg/ml of each monoclonal anti-bovine CD40 antibody. 24 hours later, cell culture supernatants were collected and assayed for the presence of bovine IL-12 by ELISA. Isotype control antibodies (Ctrl Abs) and heat-inactivated E1 antibodies were used as negative controls. LPS was used as positive control of bovine DC activation. *, P<0.05 versus unstimulated DCs.

FIG. 10: Immunization with HKSA and E1 monoclonal anti-bovine CD40 antibodies induces specific IFN-γ-secreting CD8⁺ T cells in PLN cells. Six healthy Holstein heifers were injected subcutaneously in the right prescapular region with 10⁹ CFUs of HKSA and 5 mg of the E1 monoclonal antibody (HKSA/E1 group). Control heifers received HKSA alone (10⁹ CFUs; HKSA group; n=6) or were left untreated (n=6). (A) Five days after immunization, PLN cells were isolated and restimulated in vitro for 3 days with 1×10⁵ HKSA or 50 μg/ml OVA. The proliferation was measured as ³H-thymidine incorporation during the last 16 hr of culture (upper panel). Culture supernatants were assayed for IFN-γ and IL-4 by ELISA (middle and lower panels). *, P<0.05 versus results obtained with the control cows, (B) Alternatively, PLN cells were stimulated in vitro with 1×10⁵ HKSA/ml for 20 hours and incubated with Brefeldin A for the last 4 hours. Cells were the stained for either bovine CD4 or CD8, fixed and permeabilized, and finally stained for intracellular bovine IFN-γ. Percentages of positive cells are given in the gates.

FIG. 11: HKSA/αCD40 immunization induces a Th1 type humoral response. Naive C57BL/6 mice were injected s.c. between the L4 and R4 abdominal mammary glands with 5×10⁸ HKSA and immediately afterward were given 25 μg αCD40 i.p. (HKSA/αCD40 group). Control mice received either HKSA alone (5×10⁸; s.c.), αCD40 alone (25 μg; i.p.), or PBS (100 μl s.c. and i.p.). Five days after immunization, blood was recovered and sera of each mice were evaluated for Ag-specific IgG2a and IgG2b titers by ELISA.*, significantly different from the other values (Mann-Whitney U-test, P<0.01).

FIG. 12: Map of the pDEST15 vector coding for the different forms of CD40L.

FIG. 13: Western-blot using anti-GST antibodies, showing the different forms of GST fusion-CD40L proteins produced by BL21-A1 cells under arabinose stimulation.

FIG. 14: Coomassie blue-stained gel showing the purified soluble extracellular form of CD40L preceded by the trimerization motif and fusionned to GST (i.e. Isol_solEC) and the purified GST protein alone (i.e. -) in comparison with unpurified Isol_solEC form.

FIG. 15: ELISA anti-GST measuring the relative production of proteins, in this case the soluble extracellular form of CD40L preceded by a trimerization motif (isol_solEC).

FIG. 16: ELISA anti-MCP-1 measuring the production of MCP-1 in culture supernatant of bovine aortic endothelial cells stimulated with either the eluate containing the soluble extracellular form of CD40L preceded by a trimerization motif (isol_solEC) in comparison with the same eluate which has been boiled (boiled isol_solEC), or for control eluate containing GST proteins (GST control) in comparison with the same eluate which has been boiled (boiled GST control).

FIG. 17: Graph showing the fluorescence intensity of Cos7 cells transfected with plasmids coding for bovine CD40 (black line) or not (grey line) and incubated with GST proteins (dotted line) or GST-fusion CD40L proteins (continuous line).

FIG. 18: Map of the pcDNA3.1 vector coding for the different forms of CD40L fusionned or not with GST and preceded by the signal peptide of preprotrypsinogen and a FLAG sequence.

FIG. 19: Map of the pFLAG vector coding for the different forms of CD40L fusionned or not with GST; the pFLAG vector already contains the signal peptide of preprotrypsinogen and a FLAG sequence.

FIG. 20: Detection of the different forms of CD40L proteins produced by Cos7 cells after transfection with the expression vectors pcDNA3.1 or pFLAG. A, anti-GST ELISA using anti-GST detection antibodies; B, anti-FLAG ELISA using anti-ECform detection antibodies; C, anti-FLAG ELISA using anti-GST detection antibodies.

FIG. 21: ELISA MCP-1 performed on culture medium of bovine endothelial cells stimulated with the supernatant of Cos7 cells transfected with the expression vectors pcDNA3.1 or pFLAG coding for different forms of CD40L.

FIG. 22: ELISA MCP-1 performed on culture medium of bovine endothelial cells stimulated with boiled or non-boiled supernatant of Cos7 cells transfected with the expression vectors pcDNA3.1 or pFLAG coding for different forms of CD40L.

FIG. 23: ELISA MCP-1 performed when Triton x-114 is used instead of Triton x-100 during the purification process of the proteins.

### DETAILED DESCRIPTION OF THE PREFERRED EMBODIMENTS

To reduce the negative impact of S. aureus infections and to avoid this intracellular persistence, the present invention has surprisingly found that the vaccine strategy can be based on the induction of a strong cytotoxic T lymphocyte (CTL) response. To achieve this kind of immune response in vivo, as exemplified in the appended examples, the present invention uses agonistic anti-CD40 monoclonal antibodies (mAbs) to polarize the immune response towards a CTL response against S. aureus. The results provided herein surprisingly demonstrate that immunization of mice with heat-killed S. aureus (HKSA) together with agonistic anti-CD40 mAbs elicits strong CTL responses capable of protecting mice from subsequent staphylococcal mastitis. Although the bovine CD40 receptor has been cloned and sequenced, there is currently no agonistic anti-bovine CD40 antibody available. Therefore, the present invention provides eight different anti-bovine CD40 antibodies able to specifically recognize the bovine CD40 protein. One of these antibodies displayed agonistic properties, as attested by its ability to induce a strong CTL response in vivo against S. aureus. Consequently, it has been demonstrated that this agonistic anti-CD40 antibody can be used as an adjuvant to induce CTL responses against S. aureus infections or others intracellular pathogens in cattle.

CD40 ligand (CD40L) can also be used instead of the anti-CD40 antibody. Bovine CD40L can be used in any vaccine against infectious pathogens against which a cytotoxic response is necessary. These pathogens could be virus, bacteria, fungi or parasites. Moreover, bovine CD40L could be used for any treatment where the activation of bovine CD40 receptor is required.

CD40, a member of the tumor necrosis factor receptor family, was initially characterized as a B cell surface antigen critically involved in T cell-dependent humoral immune responses. It is now known to be expressed on a wide range of cell types, including antigen-presenting cells (APCs) such as dendritic cells (DCs) and macrophages. CD40 signaling in APCs leads to the induction of robust CD4-independent CTL responses [5]. CD40 stimulation was therefore used to promote CD8⁺ T cell-mediated immunity against tumor cells and some intracellular pathogens such as Listeria monocytogenes and Leishmania major [6]. As described in detail below, the present invention surprisingly found that such an approach can be used to induce resistance to S. aureus mastitis. The results presented below demonstrate that immunization of mice with heat-killed S. aureus (HKSA) together with agonistic anti-CD40 monoclonal antibodies (mAbs) elicits strong CTL responses capable of protecting mice from subsequent staphylococcal mastitis. Our study shows promise for CTL-dependent vaccination against S. aureus mastitis.

As already mentioned above, the term “mastitis” is known to the person skilled in the art and relates to the inflammation of mammary gland tissue. S. aureus is the most common etiological organism responsible, but, among others, S. epidermidis and streptococci are occasionally isolated as well. Accordingly, mastitis consists in an inflammatory reaction of the udder tissue in cows and can become persistent. This potentially fatal mammary gland infection is the most common disease in dairy cattle worldwide. It is also the most costly to the dairy industry. Milk from cows suffering from mastitis has an increased somatic cell count, and can pose a health risk. During mastitis, white blood cells (leucocytes) are released into the mammary gland, usually in response to an invasion of bacteria of the teat canal. Milk-secreting tissue, and various ducts throughout the mammary gland are damaged due to toxins produced by the bacteria. Mastitis can also occur as a result of chemical, mechanical, or thermal injury. Mastitis can be identified by abnormalities in the udder such as swelling, heat, redness or pain. Other indications of mastitis may be abnormalities in milk such as a watery appearance, flakes, clots, or pus. Bacteria that are known to cause mastitis include:

Pseudomonas aeruginosa

Staphylococcus aureus

Staphylococcus epidermidis

Streptococcus agalactiae

Brucella melitensis

Corynebacterium bovis

Mycoplasma (various species)

Escherichia coli, (E. coli)

Klebsiella pneumoniae

Klebsiella oxytoca

Enterobacter aerogenes

Pasteurella spp.

Arcanobacterium pyogenes

Proteus spp.

Prototheca zopfii (achlorophyllic algae)

Prototheca wickerhamii (achlorophyllic algae)

Mastitis is most often transmitted by contact with the milking machine, and through contaminated hands or materials. Mastitis can cause a decline in potassium and lactoferrin. It also results in decreased casein, the major protein in milk. As most calcium in milk is associated with casein, the disruption of casein synthesis contributes to lowered calcium in milk. The milk protein continues to undergo further deterioration during processing and storage. As already mentioned above, mastitis is the most costly disease affecting dairy cattle worldwide due to the reduction in both quantity and quality of the milk produced and to the cost of treatment and control plan. E.g., this disease costs the US dairy industry about 1.7 to 2 billion USD each year. At present, treatment is possible with long-acting antibiotics, but milk from such cows is not marketable until drug residues have left the cow's system. Antibiotics may be systemic (injected into the body), or they may be forced upwards into the teat through the milk pore. Practices such as good nutrition, proper milking hygiene, and the culling of chronically infected cows can help in the control of this disease. Antibiotics like amoxicillin, and numerous other antibiotics well-known in the art are used to treat mastitis. Moreover, some S. aureus strains isolated from bovine mastitis are resistant to antibiotic therapy and are called “methicillin-resistant S. aureus or MRSA”.

Accordingly, the present invention is based on the combination of an adjuvant directed against the bovine CD40 receptor and heat killed S. aureus (HKSA). The agonistic anti-bovine CD40 mAbs is advantageously more cost-effective than the production of recombinant bovine CD40 ligand (CD40L). Even more cost effectiveness can be achieved through a DNA vaccine adjuvant, i.e. a vector containing nucleic acids codes for the anti-CD40 antibody or the CD40L. Moreover, HKSA, the antigen, is a killed whole-cell bacteria preparation and has the advantage of low cost of production. In addition, the significant antigen variation among the different strains of S. aureus responsible for mastitis has to be considered as an important factor in the development of a protective vaccine. In our vaccine strategy, integrating various HKSA strains in the same vaccine could easily circumvent this problem. Finally, S. aureus is frequently localized inside the bovine mammary cells during chronic and subclinical mastitis and this capacity of invading host cells allows S. aureus to avoid the bovine humoral immune response and the antibiotics. Our vaccine approach is the only one based on the elimination of S. aureus infected cells by using a new bovine adjuvant that polarizes the immune response towards a cytotoxic response against S. aureus.

In case of the DNA vaccine adjuvant, the strategies used to deliver DNA adjuvant could consist in intramuscular, intradermal, intra-mammary or mucosal injection using liposome-mediated delivery, gene gun or jet injection. The injected quantity should be in a range of 1 μg to 10 μg of plasmids per kg of body weight. These plasmids could be combined, or not, with a dose of CD40L proteins in a range of 1 μg to 10 μg/kg of body weight (CD40L proteins and plasmids coding for CD40L constitute the adjuvant part of the vaccine). The same strategy could also be used for plasmids containing coding for anti-CD40 monoclonal antibody. The DNA adjuvant is combined with the antigenic part of the vaccine that is for example heat-killed S. aureus (HKSA) that should be in a range of 10 to 10¹²CFU. The doses will depend on the injection site. The vaccine may be in solid, liquid or gaseous form and may be, inter alia, in a form of (a) powder(s), (a) tablet(s), (a) solution(s) or (an) aerosol(s). The CD40L proteins or plasmids coding for CD40L could also be adsorbed on microbeads. The vaccinal strategy consists in injections before and after calving.

Accordingly, as already mentioned above, the present invention relates to a combination of an agonistic anti-CD40 monoclonal antibody or CD40L or a vector coding for the anti-CD40 antibody or a vector coding for the CD40L, and inactivated or attenuated bacteria selected from the group consisting of Staphylococcus, Streptococcus, Listeria or Escherichia for use in the treatment and/or prevention of mastitis.

The combination of an agonistic anti-CD40 monoclonal antibody or CD40L or a vector coding for the anti-CD40 antibody or a vector coding for the CD40L, and inactivated or attenuated bacteria selected from the group consisting of Staphylococcus, Streptococcus, Listeria or Escherichia as defined herein above and below can, preferably, be applied in the form of a vaccine.

Moreover, without being bound by theory, the present invention does not only relate to the treatment and/or prevention of mastitis but also, generally, to the treatment and/or prevention of infectious diseases targeting other mucosae. In other words, it is also envisaged to treat or prevent infectious diseases of mucosal tissues and/or organs with the combination of the invention, i.e. the combination of an agonistic anti-CD40 monoclonal antibody or CD40L or a vector coding for the anti-CD40 antibody or a vector coding for the CD40L, and inactivated or attenuated bacteria selected from the group consisting of Staphylococcus, Streptococcus, Listeria or Escherichia. In the context of the present invention, the term mucosa, mucosal tissue and/or organs relates to a mucus-secreting membrane lining all body cavities or passages that communicate with the exterior. In the context of the present invention the following mucus-secreting membranes are of the following nature: oral, oesophageal, gastric, intestinal, nasal, bronchial, respiratory and genital mucosae, and conjunctiva. As mentioned, without being bound by theory, the present invention does not only relate to the treatment and/or prevention of mastitis but also, generally, to the treatment and/or prevention of infectious diseases targeting other mucosae. As examples only, the following infectious diseases targeting the mucosae, in, e.g., cattle, are BRS (bovine respiratory syncytial virus), IBR (infectious bovine rhinotracheitis), PI-3 (parainfluenza-3), adenoviruses, Mannheimia haemolytica and Histophilus somni for the respiratory tract; BVD (bovine viral diarrhoea), rotaviruses, coronaviruses, Escherichia coli and clostridia for the gastro-intestinal tract. In a preferred embodiment, the infectious disease of mucosal tissue to be targeted with the combination of the present invention is an infection of the mammary gland, i.e. mastitis.

Aggregation of CD40 molecules following interaction with CD40 ligand (CD40L) or CD40 specific antibodies are an important initiating step for CD40-mediated signaling. Furthermore, aggregation or clustering of CD40L is a prerequisite for the subsequent CD40 multimerization in order to initiate the intracellular signaling cascade.

Therefore, the present invention also provides a composition of a CD40 ligand (CD40L), which is capable to stimulate the CD40 receptor, in combination with inactivated or attenuated bacteria selected from the group consisting of Staphylococcus, Streptococcus, Listeria or Escherichia for use in the treatment and/or prevention of mastitis.

Preferably, the soluble form of CD40L, i.e. the extracellular domain CD40L, or a biological active fragment thereof, is used in the composition of the present invention. With the term “biological active fragment thereof” in this context soluble fragments of the extracellular domain of CD40L are encompassed which are still capable to induce aggregation of its cognate receptor, i.e. CD40. Yet, in another preferred embodiment, the soluble form of CD40L is stabilised in order to increase the stability of the trimeric form of said molecules. Appropriate methods for trimerizing proteins are well-known in the art and encompass fusion proteins, e.g. by addition of a trimerizing leucine zipper to the N-terminus of the soluble form of the CD40L.

In certain applications of the composition of the present invention the human extracellular domain of CD40L having amino acids 47-261 or amino acids 46-261 of SEQ ID No: 26, or a biological active fragment thereof as defined above is used in the composition of the present invention, preferably a fragment having amino acids 113 to 261 of SEQ ID NO:26.

SEQ ID NO: 26:
MIETYNQTSPRSAATGLPISMKIFMYLLTVFLITQMIGSALFAVYLHRRL
DKIEDERNLHEDFVFMKTIQRCNTGERSLSLLNCEEIKSQFEGFVKDIML
NKEETKKENSFEMQKGDQNPQIAAHVISEASSKTTSVLQWAEKGYYTMSN
NLVTLENGKQLTVKRQGLYYIYAQVTFCSNREASSQAPFIASLCLKSPGR
FERILLRAANTHSSAKPCGQQSIHLGGVFELQPGASVFVNVTDPSQVSHG
TGFTSFGLLKL

In an even more preferred embodiment of the present invention, the bovine form of the extracellular domain of CD40L, having amino acids 47 to 261 or amino acids 46-261 of the complete bos taurus CD40L sequence as shown in SEQ ID NO:27, or a biological active fragment thereof as defined above, e.g. a fragment having amino acids 113 to 261 of SEQ ID NO:27 is used in the composition of the present invention.

SEQ ID NO: 27:
MIETYSQPSPRSVATGPPVSMKIFMYLLTVFLITQMIGSALFAVYLHRRL
DKIEDERNLHEDFVFMKTIQRCNKGEGSLSLLNCEEIRSRFEDLVKDIMQ
NKEVKKKEKNFEMHKGDQEPQIAAHVISEASSKTTSVLQWAPKGYYTLSN
NLVTLENGKQLAVKRQGFYYIYTQVTFCSNRETLSQAPFIASLCLKSPSG
SERILLRAANTHSSSKPCGQQSIHLGGVFELQSGASVFVNVTDPSQVSHG
TGFTSFGLLKL

In context of the present invention, the term “antibody” or “antibody molecule” relates to full immunoglobulin molecules, preferably IgMs, IgDs, IgEs, IgAs or IgGs, more preferably IgG1, IgG2, IgG2b, IgG3 or IgG4 as well as to parts of such immunoglobulin molecules. Furthermore, the term relates to modified and/or altered antibody molecules, like chimeric and bovinized or humanized antibodies. In a preferred embodiment, the antibody is bovinized. The term also relates to monoclonal or polyclonal antibodies as well as to recombinantly or synthetically generated/synthesized antibodies. In a preferred embodiment, the anti-CD40 antibody is a monoclonal antibody. The term also relates to intact antibodies as well as to antibody fragments thereof, like, separated light and heavy chains, Fab, Fab/c, Fv, Fab′, F(ab′)₂. The term “antibody molecule” also comprises bifunctional antibodies and antibody constructs, like single chain Fvs (scFv) or antibody-fusion proteins. Further details on the term “antibody molecule” of the invention are provided herein below.

Techniques for the production of antibodies are well known in the art and described, e.g. in Howard and Bethell (2000) Basic Methods in Antibody Production and Characterization, Crc. Pr. Inc. Antibodies directed against a polypeptide or a protein according to the present invention (i.e. CD40) can be obtained, e.g., by direct injection of the polypeptide (or a fragment thereof) into an animal or by administering the polypeptide (or a fragment thereof) to an animal. The antibody so obtained will then bind polypeptide (or a fragment thereof) itself. In this manner, even a fragment of the polypeptide can be used to generate antibodies binding the whole polypeptide, as long as said binding is “specific” as defined above.

These polypeptides are particularly useful in the preparation of specific antibodies and are provided herein for illustrative purposes.

With the normal skill of the person skilled in the art and by routine methods, the person skilled in the art can easily deduce from the sequences provided herein relevant epitopes (also functional fragments) of the polypeptides of the present invention which are useful in the generation of antibodies like polyclonal and monoclonal antibodies. However, the person skilled in the art is readily in a position to also provide for engineered antibodies like CDR-grafted antibodies or also bovinized or humanized and fully human or bovine antibodies and the like.

Particularly preferred in the context of the present invention are monoclonal antibodies. For the preparation of monoclonal antibodies, any technique which provides antibodies produced by continuous cell line cultures can be used. Examples for such techniques include the hybridoma technique, the trioma technique, the human B-cell hybridoma technique and the EBV-hybridoma technique to produce human monoclonal antibodies (Shepherd and Dean (2000), Monoclonal Antibodies: A Practical Approach, Oxford University Press, Goding and Goding (1996), Monoclonal Antibodies: Principles and Practice—Production and Application of Monoclonal Antibodies in Cell Biology, Biochemistry and Immunology, Academic Pr Inc, USA).

The antibody derivatives can also be produced by peptidomimetics. Further, techniques described for the production of single chain antibodies (see, inter alia, U.S. Pat. No. 4,946,778) can be adapted to produce single chain antibodies specifically recognizing the polypeptide of the invention. Also, transgenic animals may be used to express humanized or bovinized antibodies to the polypeptide of the invention.

The present invention also envisages the production of a specific antibody against native polypeptides and recombinant polypeptides according to the invention. This production is based, for example, on the immunization of animals, like mice. However, also other animals for the production of antibody/antisera are envisaged within the present invention. For example, monoclonal and polyclonal antibodies can be produced by rabbit, mice, goats, donkeys and the like. The polynucleotide of CD40 according to the invention and as described below can be subcloned into an appropriated vector, wherein the recombinant polypeptide is to be expressed in an organism being able for an expression, for example in bacteria. Thus, the expressed recombinant protein can be intra-peritoneally injected into a mice and the resulting specific antibody can be, for example, obtained from the mice serum being provided by intra-cardiac blood puncture. The present invention also envisages the production of specific antibodies against native polypeptides and recombinant polypeptides by using a DNA vaccine strategy as exemplified in the appended examples. DNA vaccine strategies are well-known in the art and encompass liposome-mediated delivery, by gene gun or jet injection and intramuscular or intradermal injection. Thus, antibodies directed against a polypeptide or a protein according to the present invention (i.e. CD40) can be obtained by directly immunizing the animal by directly injecting intramuscularly the vector expressing the CD40, in particular the bovine CD40. The amount of obtained specific antibody can be quantified using an ELISA, which is also described herein below. Further methods for the production of antibodies are well known in the art, see, e.g. Harlow and Lane, “Antibodies, A Laboratory Manual”, CSH Press, Cold Spring Harbor, 1988.

The term “specifically binds”, as used herein, refers to a binding reaction that is determinative of the presence of the CD40 protein and antibody in the presence of a heterogeneous population of proteins and other biologics.

Thus, under designated assay conditions, the specified antibodies and CD40 proteins bind to one another and do not bind in a significant amount to other components present in a sample. Specific binding to a target analyte under such conditions may require a binding moiety that is selected for its specificity for a particular target analyte. A variety of immunoassay formats may be used to select antibodies specifically reactive with a particular antigen. For example, solid-phase ELISA immunoassays are routinely used to select monoclonal antibodies specifically immunoreactive with an analyte. See Shepherd and Dean (2000), Monoclonal Antibodies: A Practical Approach, Oxford University Press and/or Howard and Bethell (2000) Basic Methods in Antibody Production and Characterization, Crc. Pr. Inc. for a description of immunoassay formats and conditions that can be used to determine specific immunoreactivity. Typically a specific or selective reaction will be at least twice background signal to noise and more typically more than 10 to 100 times greater than background. The person skilled in the art is in a position to provide for and generate specific binding molecules directed against the novel polypeptides. For specific binding-assays it can be readily employed to avoid undesired cross-reactivity, for example polyclonal antibodies can easily be purified and selected by known methods (see Shepherd and Dean, loc. cit.).

The term “anti-CD40 antibody” means in accordance with this invention that the antibody molecule is capable of specifically recognizing or specifically interacting with and/or binding to at least two amino acids of each of the two regions of CD40 as defined herein. Said term relates to the specificity of the antibody molecule, i.e. to its ability to discriminate between the specific regions of CD40 peptide as defined herein. Accordingly, specificity can be determined experimentally by methods known in the art and methods as disclosed and described herein. Such methods comprise, but are not limited to Western blots, ELISA-, RIA-, ECL-, IRMA-tests and peptide scans. Such methods also comprise the determination of K_{D}-values as, inter alia, illustrated in the appended examples. The peptide scan (pepspot assay) is used routinely employed to map linear epitopes in a polypeptide antigen. The primary sequence of the polypeptide is synthesized successively on activated cellulose with peptides overlapping one another. The recognition of certain peptides by the antibody to be tested for its ability to detect or recognize a specific antigen/epitope is scored by routine colour development (secondary antibody with horseradish peroxide and 4-chloronaphtol and hydrogenperoxide), by a chemoluminescence reaction or similar means known in the art. In the case of, inter alia, chemoluminescence reactions, the reaction can be quantified. If the antibody reacts with a certain set of overlapping peptides one can deduce the minimum sequence of amino acids that are necessary for reaction. The same assay can reveal two distant clusters of reactive peptides, which indicate the recognition of a discontinuous, i.e. conformational epitope in the antigenic polypeptide (Geysen (1986), Mol. Immunol. 23, 709-715).

In the context of the present invention, the term “CD40” relates to the protein CD40 which is a 45-50 kDa transmembrane receptor that belongs to the tumor necrosis factor-α (TNF-α) receptor family. This receptor is expressed on the surface of dendritic cells (DCs), macrophages, epithelial cells, hematopoietic progenitors, and activated T cells. CD40 plays a pivotal role in governing cell mediated immunity, mainly manifested by the activation of DCs [1]. Indeed, recent evidence in mice indicates that stimulating DCs in vivo with agonistic anti-CD40 monoclonal antibodies allows induction of robust CTL responses against concomitantly administered antigens. Engagement of CD40 on the surface of DCs results in cellular maturation and activation, including up-regulation of surface co-stimulatory molecules and secretion of inflammatory cytokines including interleukin (IL)-12, all of which are required for CTL induction [1].

The coding regions of the CD40 as disclosed herein or functional fragments thereof are known in the art and comprise, inter alia, the GenBank entries for Homo sapiens, NG_{—}007279.1; Canis lupus, NM_{—}001002982.1; Equus caballus, NM_{—}001081902.1; Bos taurus, NM_{—}001105611.1; Rattus norvegicus, NM_{—}134360.1; and Gallus gallus, NM_{—}204665.1. The person skilled in the art may easily deduce the relevant coding region of the CD40 as disclosed in these GenBank entries, which may also comprise the entry of genomic DNA as well as mRNA/cDNA.

The bovine CD40 cDNA is given in SEQ ID NO:17. Thus, in particular, bovine CD40 may be encoded by the following nucleic acid sequence:

SEQ ID NO: 17
ATGGTTCGTTTGCCACTGCAGTGTCTCTTCTGGGGCTTCTTTCTGACCGC
CGTCCACTCAGAACCAGCCACTGCTTGTGGAGAGAAGCAATACCCAGTGA
ACAGTCTTTGCTGTGATTTGTGCCCGCCGGGACAGAAACTGGTGAACGAC
TGCACAGAGGTCAGCAAAACAGAATGCCAGTCCTGCGGTAAAGGCGAATT
CTTGTCCACCTGGAACAGAGAGAAATACTGTCACGAGCACAGATACTGCA
ACCCCAACCTAGGGCTCCGGATCCAGAGCGAGGGTACCTTGAATACAGAC
ACCATTTGTGTATGTGTCGAAGGCCAACACTGTACCAGTCACACCTGCGA
AAGTTGCACGCCCCACAGCTTGTGTCTCCCTGGCTTCGGGGTCAAGCAGA
TCGCTACAGGGCTTTTGGATACCGTCTGTGAACCCTGCCCGCTCGGCTTC
TTCTCCAACGTGTCATCTGCTTTTGAAAAGTGTCACCGTTGGACAAGCTG
CGAGAGAAAAGGCCTGGTGGAACAACACGTGGGGACGAACAAGACAGATG
TTGTCTGCGGTTTCCAGAGTCGGATGAGGACCCTGGTGGTGATCCCCGTC
ACGATGGGAGTCTTGTTTGCTGTCCTGTTGGTATCTGCCTGTATCAGGAA
CATAACCAAGAAGCGGCAGGCTAAGGCCCTGCACCCTATGGCTGAAAGGC
AGGATCCCGTGGAGACGATTGATCCGGAGGATTTTCCCGGCCCCCACCCG
CCTCCTCCGGTGCAAGAGACCTTATGCTGGTGTCAGCCGGTCGCCCAGGA
GGACGGCAAAGAGAGCCGCATCTCCGTGCAGGAGCGAGAATGA

which corresponds to the following amino acid sequence: SEQ ID NO:18

MVRLPLQCLFWGFFLTAVHSEPATACGEKQYPVNSLCCDLCPPGQKLVND
CTEVSKTECQSCGKGEFLSTWNREKYCHEHRYCNPNLGLRIQSEGTLNTD
TICVCVEGQHCTSHTCESCTPHSLCLPGFGVKQIATGLLDTVCEPCPLGF
FSNVSSAFEKCHRWTSCERKGLVEQHVGTNKTDVVCGFQSRMRTLVVIPV
TMGVLFAVLLVSACIRNITKKRQAKALHPMAERQDPVETIDPEDFPGPHP
PPPVQETLCWCQPVAQEDGKESRISVQERE

It is particularly preferred that the antibody molecule of the invention comprises a variable V_{H}-region as encoded by a nucleic acid molecule as shown in

### SEQ ID NO:7:

GAAGTGAAGCTGGTGGAGTCTGGGGGAGTCTTAGTGAAGCCTGGAGGGTC
CCTGAAACTCTCCTGTGCAGCCTCTGGATTCACTTTCAGTAGCTATGCCA
TGTCTTGGGTTCGCCAGACTCCAGAAAAGAGGCTGGAGTGGGTCGCATCT
ATTGGTAGTGGTGGTGGAACTTACTATCCAGACAGTGTGAAGGGCCGATT
CACCATCTCCAGAAATAATGCCAGGAACATCCTGTACCTGCAAATGAGCA
GTCTGAGGTCTGAGGACACGGCCATGTATTACTGTGCAAGAGCCTACTAT
AGGAACCACCGAGGGTCTGTTATGGACTACTGGGGTCAAGGAACCTCAGT
CACCGTCTCCTCA

or a variable V_{H}-region as shown in the amino acid sequences depicted in SEQ ID NO:8:

EVKLVESGGVLVKPGGSLKLSCAASGFTFSSYAMSWVRQTPEKRLEWVAS
IGSGGGTYYPDSVKGRFTISRNNARNILYLQMSSLRSEDTAMYYCARAYY
RNHRGSVMDYWGQGTSVTVSS.

The sequences as shown in SEQ ID NO:7 and 8 depict the coding region and the amino acid sequence of the V_{H}-region of the inventive, monoclonal anti-CD40 antibody. Accordingly, the invention also provides for antibody molecules which comprise a variable V_{L}-region as encoded by a nucleic acid molecule as shown in a SEQ ID NO:9:

GACATTGTGCTGACCCAATCTCCAGCTTCTTTGGCTGTGTCTCTAGGGCA
GAGGGCCACCATCTCCTGCAAGGCCAGCCAAACTGTTGATTATGATGGTG
ATAGTTATATGAACTGGTACCAACAGAAACCAGGACAGCCACCCAAAGTC
CTCATCTATTCTGCATCCAATCTGGAATCTGGGATCCCAGCCAGGTTTAG
TGGCAGTGGGTCTGGGACAGACTTCACCCTCAACATCCATCCTGTGGAGG
AGGAGGATGCTGCAACCTATTACTGTCAGCAAAGTACTGAGGATCCTCCG
ACGTTCGGTGGAGGCACCAAGCTGGAAATCAAA.

It is preferred that the antibodies/antibody molecules of the invention are characterized by their specific reactivity with CD40 and/or peptides derived from said CD40. For example, optical densities in ELISA-tests, may be established and the ratio of optical densities may be employed to define the specific reactivity of the antibodies. Accordingly, a preferred antibody of the invention is an antibody which reacts in an ELISA-test with CD40 to arrive at an optical density measured at 450 nm that is 10 times higher than the optical density measured without CD40, i.e. 10 times over background. If the optical reading is performed after a certain time, e.g. 5 minutes, a signal over background ratio of 10 is obtained with the antibodies of the present invention.

In a particular preferred embodiment, the inventive agonistic anti-CD40 antibody molecule comprises at least one CDR′ of an V_{L}-region as encoded by a nucleic acid molecule as shown in SEQ ID NO:9:

GACATTGTGCTGACCCAATCTCCAGCTTCTTTGGCTGTGTCTCTAGGGCA
GAGGGCCACCATCTCCTGCAAGGCCAGCCAAACTGTTGATTATGATGGTG
ATAGTTATATGAACTGGTACCAACAGAAACCAGGACAGCCACCCAAAGTC
CTCATCTATTCTGCATCCAATCTGGAATCTGGGATCCCAGCCAGGTTTAG
TGGCAGTGGGTCTGGGACAGACTTCACCCTCAACATCCATCCTGTGGAGG
AGGAGGATGCTGCAACCTATTACTGTCAGCAAAGTACTGAGGATCCTCCG
ACGTTCGGTGGAGGCACCAAGCTGGAAATCAAA

or at least one CDR amino acid sequence of an V_{L}-region as shown in SEQ ID NO:10:

DIVLTQSPASLAVSLGQRATISCKASQTVDYDGDSYMNWYQQKPGQPPKV
LIYSASNLESGIPARFSGSGSGTDFTLNIHPVEEEDAATYYCQQSTEDPP
TFGGGTKLEIK

and/or said antibody molecule comprises at least one CDR of an V_{H}-region as encoded by a nucleic acid molecule as shown in SEQ ID NO:7:

GAAGTGAAGCTGGTGGAGTCTGGGGGAGTCTTAGTGAAGCCTGGAGGGTC
CCTGAAACTCTCCTGTGCAGCCTCTGGATTCACTTTCAGTAGCTATGCCA
TGTCTTGGGTTCGCCAGACTCCAGAAAAGAGGCTGGAGTGGGTCGCATCT
ATTGGTAGTGGTGGTGGAACTTACTATCCAGACAGTGTGAAGGGCCGATT
CACCATCTCCAGAAATAATGCCAGGAACATCCTGTACCTGCAAATGAGCA
GTCTGAGGTCTGAGGACACGGCCATGTATTACTGTGCAAGAGCCTACTAT
AGGAACCACCGAGGGTCTGTTATGGACTACTGGGGTCAAGGAACCTCAGT
CACCGTCTCCTCA

or at least one CDR amino acid sequence of an V_{H}-region as shown in SEQ ID NO:8:

EVKLVESGGVLVKPGGSLKLSCAASGFTFSSYAMSWVRQTPEKRLEWVAS
IGSGGGTYYPDSVKGRFTISRNNARNILYLQMSSLRSEDTAMYYCARAYY
RNHRGSVMDYWGQGTSVTVSS.

Consequently, in accordance with the above, the CDRs of an V_{L}-region are selected from the group consisting of CDR1′ as encoded by a nucleic acid molecule as shown in SEQ ID NO: 11:

AAGGCCAGCCAAACTGTTGATTATGATGGTGATAGTTATATGAAC

or CDR1′ amino acid sequence as shown in SEQ ID NO:4:

KASQTVDYDGDSYMN,

CDR2′ as encoded by a nucleic acid molecule as shown in SEQ ID NO:12:

TCTGCATCCAATCTGGAATCT

or CDR2′ amino acid sequence as shown in SEQ ID NO:5:

SASNLES

and CDR3′ as encoded by a nucleic acid molecule as shown in SEQ ID NO:13:

CAGCAAAGTACTGAGGATCCTCCGACG

or CDR3′ amino acid sequence as shown in SEQ ID NO:6:

QQSTEDPPT.

Consequently, in accordance with the above, the CDRs of an V_{H}-region are selected from the group consisting of CDR1 as encoded by a nucleic acid molecule as shown in SEQ ID NO:14:

AGCTATGCCATGTCT

or CDR1 amino acid sequence as shown in SEQ ID NO:1:

SYAMS,

CDR2 as encoded by a nucleic acid molecule as shown in SEQ ID NO:15:

TCTATTGGTAGTGGTGGTGGAACTTACTATCCAGACAGTGTGAAGGGC

or CDR2 amino acid sequence as shown in SEQ ID NO2:

SIGSGGGTYYPDSVKD and

CDR3 as encoded by a nucleic acid molecule as shown in SEQ ID NO:16:

GCCTACTATAGGAACCACCGAGGGTCTGTTATGGACTAC

or CDR3 amino acid sequence as shown in SEQ ID NO3:

AYYRNHRGSVMDY.

Most preferred, the antibody of the invention comprises at least one CDR and/or CDR′, more preferred at least two CDR and/or CDR′ and most preferred all three CDR and/or CDR′.

In a more preferred embodiment of the invention, the antibody molecule is a full antibody (immunoglobulin, like an IgG1, an IgG2, an IgG2b, an IgG3, an IgG4, an IgA, an IgM, an IgD or an IgE), an F(ab)-, Fabc-, Fv-, Fab′-, F(ab′)₂-fragment, a single-chain antibody, a chimeric antibody, a CDR-grafted antibody, a bivalent antibody-construct, an antibody-fusion protein or a synthetic antibody.

The inventive antibodies/antibody molecules can readily be recombinantly constructed and expressed. Preferably, the antibody molecule of the invention comprises at least one, more preferably at least two, preferably at least three, more preferably at least four, more preferably at least five and most preferably at least six CDRs of the herein defined antibodies. The person skilled in the art can readily employ the information given herein to deduce corresponding CDRs of the antibodies.

Inventive antibody molecules can easily be produced in sufficient quantities, inter alia, by recombinant methods known in the art, see, e.g. Bentley, Hybridoma 17 (1998), 559-567; Racher, Appl. Microbiol. Biotechnol. 40 (1994), 851-856; Samuelsson, Eur. J. Immunol. 26 (1996), 3029-3034.

The term “agonistic” as used herein is known in the art and relates to a molecule capable of fully or partially mimic the action of a naturally occurring substance by binding of said molecule to a receptor of a cell and triggering a response by the cell. Consequently, an agonist often mimics the action of a naturally occurring substance. Thus, an agonist produces an action and, therefore, acts in contrast to an antagonist that rather blocks an action of an agonist. In the context of the present invention, the term “agonistic” relates to an anti-CD40 monoclonal antibody that is capable of fully or partially mimics the physiologic activity of (a) specific protein(s). In the context of the present invention said agonistic anti-CD40 monoclonal antibody, therefore, may elicit strong CTL responses capable of treating or preventing or protecting a subject from mastitis in combination with inactivated or attenuated bacteria as mentioned above. The agonistic anti-CD40 monoclonal antibody is able to specifically recognize the CD40 protein. In addition, the agonistic anti-CD40 monoclonal antibody displays agonistic properties, i.e. it has the ability, as exemplified in the appended examples, to induce a strong CTL response, wherein said CTL response is capable to protect a subject from mastitis, preferably staphylococcal mastitis. This CTL response can be measured/detected by the herein described methods. As a non-limiting example for a cytotoxic test in vivo:

Spleens and lymph nodes are removed from naïve C57BL/6 mice and a single cell suspension was made. Spleen and lymph node cells were then pulsed or not with 5 μg/ml OT-I OVA peptide (chicken OVA peptide 257-264 SIINFEKL; NEOSYSTEM) and labeled with 5,6-carboxyfluorescein diacetate succinimidyl ester (CFSE; Molecular Probe). CFSE is kept as a 0.5 mM stock in DMSO and stored at −20° C. Peptide-pulsed splenocytes and lymph node cells are labeled with CFSE at a final concentration of 5 μM, whereas unpulsed cells are labeled at a 10-fold lower concentration by incubation for 10 min at 37° C. After labeling, FCS is added to a final concentration of 5% and cells are washed twice with ice-cold phosphate-buffered saline (PBS). The two cell populations are mixed at a 1:1 ratio and approximately 5×10⁷ cells are injected i.v. into recipient mice that were injected with either PBS (100 μl, s.c. and i.p.), OVA (10 μg, s.c.), αCD40 (25 i.p.), or OVA/αCD40 (10 s.c. and 25 μg, i.p., respectively) 5 days before injection of the CFSE-labeled targets. MLNs are excised 16 h later after the injection of unpulsed/pulsed cells and single cell suspensions are analyzed by flow cytometry for detection and quantification of CFSE-labeled cells. The percentage of antigen-specific lysis in vivo is calculated as follows: (1−number of CFSE^{hi} cells/number of CFSE^{lo} cells)×100.

Other well known methods for measuring the CTL response are well known to the person skilled in the art. Consequently, without being bound by theory, the induction of the CTL response by the agonistic anti-CD40 monoclonal antibody or CD40L proteins can be measured by the following method: A CTL response is characterized by the development of antigen-specific CD8+ T cells capable of producing IFN-gamma. To demonstrate that these cells have been induced, it is necessary to collect draining lymph node cells, to stimulate them ex vivo with the antigen (or an irrelevant antigen as a control to prove the specificity), and to demonstrate that CD8+ T cells producing IFN-gamma are present in the culture. These cells can be visualized by flow cytometry after double staining for the surface CD8 receptor and for intracellular IFN-γ.

Without being bound by theory, it is believed that the agonistic anti-CD40 monoclonal antibody displays agonistic properties, i.e. it has medical implications, i.e. the ability, as exemplified in the appended examples, to induce a strong CTL response, wherein said strong CTL response is capable of protecting, treating or preventing mastitis. Consequently, the induction of the CTL response is expected to have medical implications, i.e., e.g., to reduce the negative impact of infections like intracellular bovine infections caused by intracellular pathogens, i.e., e.g. mastitis. As already mentioned above, the term “mastitis” is known to the person skilled in the art. Preferably, said intracellular pathogen is S. aureus. Staphylococcus aureus, a common gram-positive bacterium, is the most prevalent infectious agent that causes subclinical mastitis. This high prevalence is partly explained by the fact that staphylococcal mastitis remains difficult to treat and/or control efficiently. This is notably related to the ability of S. aureus to invade and survive within host phagocytes and mammary epithelial cells. Indeed, intracellular invasion provides protection from the humoral immune response and several classes of antibiotics [3, 4]. To reduce the negative impact of S. aureus infections, the present invention has surprisingly found that agonistic anti-CD40 monoclonal antibody displays agonistic properties, and, as exemplified in the appended examples, avoids intracellular persistence of the pathogen. Accordingly, to avoid this intracellular persistence, the vaccine strategy of the present invention is based on the induction of a strong cytotoxic T lymphocyte (CTL) response. To achieve this kind of immune response in vivo, the above mentioned agonistic anti-CD40 monoclonal antibodies (mAbs) are used to polarize the immune response towards a CTL response against S. aureus. As demonstrated in the appended examples, immunization of mice with heat-killed S. aureus (HKSA) together with agonistic anti-CD40 mAbs elicits strong CTL responses capable of protecting mice from subsequent staphylococcal mastitis. As already mentioned above, said intracellular pathogen is preferably Staphylococcus aureus (S. aureus). However, it is also envisaged to use the vaccine strategy of the present invention for other intracellular infections, preferably bovine infections, caused by other bacteria that cause mastitis as outlined above as well as other intracellular pathogens, i.e. viruses or parasites.

Accordingly, and in accordance with the above, in a preferred embodiment, said inventive agonistic anti-CD40 antibody molecule comprises at least one, more preferred two or three and even more preferred four or five and in a most preferred embodiment six of the above-defined CDR and/or CDR′ and relates to an antibody molecule that exhibits agonistic properties.

The term “inactivated or attenuated bacteria selected from the group consisting of Staphylococcus, Streptococcus, Listeria or Escherichia” is known to the person skilled in the art and relates as used herein to the inactivation or attenuation of bacterial organisms. Accordingly, an attenuated bacterium as used herein relates to a bacterial organism that is created by reducing the virulence of said bacterium by methods very well known to the skilled person, but still keeping it viable (or “live”). Hence, attenuation takes a living agent and alters it so that it becomes harmless or less virulent. In contrast to the term “attenuated bacteria”, “inactivated bacteria” relates to bacteria that are produced by “killing” said bacterial organism. Without being bound by theory, as an example, the inactivated bacteria also refers to the term “heat killed S. aureus (HKSA)” as used herein, and can be produced by the following protocol. S. aureus Newbould 305 (American Type Culture Collection 29740), a mastitis isolate, was the pathogen used. A single colony from a Nutrient (Difco Laboratories) agar plate was inoculated into 4 ml of Nutrient Broth and grown at 37° C. with vigorous shaking for 7 h. From this 4-ml culture, 100 μl were transferred into 10 ml of Nutrient Broth and incubated overnight (16 h) at 37° C. with vigorous shaking. The overnight culture was centrifuged, and the pellet was resuspended in PBS at a density of 10¹⁰ CFU/ml. Dilutions of this stock suspension can then be used. For preparation of HKSA, bacteria were incubated at 60° C. for 75 min. Effective bacterial killing was confirmed by incubating a 100-μl aliquot in Nutrient Broth overnight at 37° C. However, based on his general knowledge and the prior art, the skilled person is readily in a position to prepare inactivated or attenuated bacteria selected from the group consisting of Staphylococcus, Streptococcus, Listeria or Escherichia by using different approaches.

The term “combination” as used herein relates to a combination of an agonistic anti-CD40 monoclonal antibody or CD40 ligand (CD40L) or a vector coding for the anti-CD40 antibody or a vector coding for the CD40L, and inactivated or attenuated bacteria as described herein above. In a preferred embodiment, a simultaneous application is envisaged. Yet, the combination also encompasses a subsequent application of the two components, i.e. an agonistic anti-CD40 monoclonal antibody or CD40 ligand (CD40L) or a vector coding for the anti-CD40 antibody or a vector coding for the CD40L, and inactivated or attenuated bacteria as described herein above.

The term “treatment and/or prevention” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes the treatment of mastitis.

Nevertheless, the present invention does not only relate to the treatment and/or prevention of mastitis but also, generally, to the treatment and/or prevention of infectious diseases targeting other mucosae. In other words, it is also envisaged to treat or prevent infectious diseases of mucosal tissues and/or organs with the combination of the invention, i.e. the combination of an agonistic anti-CD40 monoclonal antibody or CD40 ligand (CD40L) or a vector coding for the anti-CD40 antibody or a vector coding for the CD40L, and inactivated or attenuated bacteria selected from the group consisting of Staphylococcus, Streptococcus, Listeria or Escherichia. In the context of the present invention, the term mucosa, mucosal tissue and/or organs relates to a mucus-secreting membrane lining all body cavities or passages that communicate with the exterior. In the context of the present invention the following mucus-secreting membranes are of the following nature: oral, oesophageal, gastric, intestinal, nasal, bronchial, respiratory and genital mucosae, and conjunctiva. As mentioned, without being bound by theory, the present invention does not only relate to the treatment and/or prevention of mastitis but also, generally, to the treatment and/or prevention of infectious diseases targeting other mucosae. As examples only, the following infectious diseases targeting the mucosae, in, e.g., cattle, are BRS (bovine respiratory syncytial virus), IBR (infectious bovine rhinotracheitis), PI-3 (parainfluenza-3), adenoviruses, Mannheimia haemolytica and Histophilus somni for the respiratory tract; BVD (bovine viral diarrhoea), rotaviruses, coronaviruses, Escherichia coli and clostridia for the gastro-intestinal tract. In a preferred embodiment, the infectious disease of mucosal tissue to be targeted with the combination of the present invention is an infection of the mammary gland, i.e. mastitis.

A “patient” or “subject” for the purposes of the present invention includes both humans and other animals, particularly mammals, and other organisms. Thus, the methods are applicable to both human therapy and veterinary applications. In a preferred embodiment the patient is a mammal, and in the most preferred embodiment the patient or subject is dairy cattle, sheep or goats.

Accordingly, in accordance with the above, and in relation with the embodiments of this invention, the present invention relates to a combination of an agonistic anti-CD40 monoclonal antibody and inactivated or attenuated bacteria selected from the group consisting of Staphylococcus, Streptococcus, Listeria or Escherichia for use in the treatment and/or prevention of mastitis, wherein the agonistic anti-CD40 monoclonal antibody comprises a) an immunoglobulin heavy chain variable domain (VH) which comprises the hypervariable regions CDR1, CDR2 and CDR3, said CDR1 having the amino acid sequence SEQ ID NO:1:

SYAMS,

said CDR2 having the amino acid sequence SEQ ID NO:2:

SIGSGGGTYYPDSVKD,

and said CDR3 having the amino acid sequence SEQ ID NO:3:

AYYRNHRGSVMDY;

b) an immunoglobulin light chain variable domain (VL) which comprises the hypervariable regions CDR1′, CDR2′ and CDR3′, said CDR1′ having the amino acid sequence SEQ ID NO:4:

KASQTVDYDGDSYMN,

said CDR2′ having the amino acid sequence SEQ ID NO:5:

SASNLES,

and said CDR3′ having the amino acid sequence SEQ ID NO:6:

QQSTEDPPT;

for use in the treatment and/or prevention of mastitis or variants thereof capable of inducing CD40 receptor aggregation.

The term “aggregation” in the phrase “capable of inducing CD40 receptor aggregation” relates to the aggregation of receptor molecules or receptor clustering. It is well-known that this is a central event in the signalling of many types of receptor molecules and is initiated by the interaction of a ligand with its cognate receptor. To determine if the anti-CD40 antibodies of the present invention are able to induce bovine CD40 aggregation, and therefore CD40 signalling, monocyte-derived bovine dendritic cells are generated, a cell type known to express large amounts of CD40. These cells are stimulated with different monoclonal antibodies and measured whether the dendritic cells were able to produce interleukin-12 (IL-12) upon stimulation. IL-12 production is indeed a classical outcome of CD40 aggregation. IL-12 was measured in the supernatant of stimulated dendritic cells by ELISA. The E1 hybridoma produced monoclonal antibodies capable of inducing IL12 production by bovine dendritic cells. The E1 antibodies were therefore the only antibodies endowed with agonistic properties.

In a further embodiment, in accordance with the above, the present invention relates to a combination of an agonistic anti-CD40 monoclonal antibody and inactivated or attenuated bacteria selected from the group consisting of Staphylococcus, Streptococcus, Listeria or Escherichia for use in the treatment and/or prevention of mastitis, wherein the agonistic anti-CD40 monoclonal antibody is an anti-bovine CD40 antibody.

In yet another further embodiment, and in accordance with the above, the present invention relates to a combination of an agonistic anti-CD40 monoclonal antibody and inactivated or attenuated bacteria selected from the group consisting of Staphylococcus, Streptococcus, Listeria or Escherichia for use in the treatment and/or prevention of mastitis, wherein the antibody is produced by the hybridoma cells deposited on Apr. 2, 2010 at the International Depositary Authority of the Belgian Coordinated Collections of Microorganisms (BCCM/LMBP) Collection, Department of Molecular Biology, Ghent University, Technologiepark 927, B-9052 Gent-Zwijnaarde, Belgium, in accordance with the Budapest Treaty of 28 Apr. 1977 on the International Recognition of the Deposit of Microorganisms for the Purposes of Patent Procedure under No: LMBP 7218CB [i.e. clone E1]. Said hybridoma cells (i.e. the clone E1) are issued from the fusion of spleen lymphocytes (isolated from mice immunized with cells transfected with a plasmid coding the bovine CD40 receptor) with myeloma cells which lacks the hypoxanthine-guanine phosphoribosyltransferase. After a selection with hypoxanthine-aminopterin-thymidine, single cells per well culture (96 wells plate) were assessed for IgG production by ELISA screening and after for specificity to bovine CD40 receptor. The clone E1 is one of the clones issued from this experiment.

In a particular embodiment of the present invention and in accordance with the above, the Staphylococcus is Staphylococcus aureus or Staphylococcus agalactiae, the Streptococcus is Streptococcus uberis, the Escherichia is Eschericha coli and the Listeria is Listeria monocytogenes.

As already mentioned above, in a further embodiment, the present invention relates to a combination of an agonistic anti-CD40 monoclonal antibody or CD40 ligand (CD40L) or a vector coding for the anti-CD40 antibody or a vector coding for the CD40L, and inactivated or attenuated bacteria selected from the group consisting of Staphylococcus, Streptococcus, Listeria or Escherichia for use in the treatment and/or prevention of mastitis in dairy cattle, sheep or goats.

As already mentioned above, in further embodiments, the combination of the agonistic anti-CD40 monoclonal antibody or CD40 ligand (CD40L) or a vector coding for the anti-CD40 antibody or a vector coding for the CD40L, as well as the inactivated or attenuated bacteria as defined above can be applied in the form of a vaccine.

The invention also provides for a nucleic acid molecule encoding an inventive antibody molecule as defined herein, i.e. nucleic acid molecule encoding the agonistic anti-CD40 monoclonal antibody of the invention, or a nucleic acid molecule encoding CD40L.

Accordingly, the agonistic anti-CD40 monoclonal antibody molecules to be employed in the context of the present invention comprise, but are not limited to the molecules encoded by the nucleic acid molecules as described herein above. Also envisaged are agonistic anti-CD40 monoclonal antibody variants or orthologs which are at least 60%, 70%, 80%, 90%, 95%, 96%, 97%, 98% or 99% identical to nucleic acid sequence as shown above. These agonistic anti-CD40 monoclonal antibody molecules as referred here are defined as molecules that are capable of acting as a functional agonistic anti-CD40 monoclonal antibody as described herein above and below. These functions and activities include, inter alia, eliciting strong CTL responses capable of treating or preventing or protecting a subject from mastitis in combination with inactivated or attenuated bacteria as mentioned above. The agonistic anti-CD40 monoclonal antibody is able to specifically recognize the CD40 protein. In addition, the agonistic anti-CD40 monoclonal antibody displays agonistic properties, i.e. it has the ability, as exemplified in the appended examples, to induce a strong CTL response, wherein said CTL response is capable to protect a subject from mastitis, preferably staphylococcal mastitis. For testing the functional agonistic anti-CD40 monoclonal antibody activity, assays provided herein below and as exemplified in the appended examples may be used. Furthermore envisaged are agonistic anti-CD40 monoclonal antibody variants or orthologs which are at least 60%, 70%, 80%, 90%, 95%, 96%, 97%, 98% or 99% identical to the amino acid sequence as shown above and being capable of acting as a functional agonistic anti-CD40 monoclonal antibody as described herein above and below. Furthermore, it is envisaged that at least one, two, three or even four amino acid residue(s) in the above-defined CDRs of the anti-CD40 monoclonal antibody variants of the invention are substituted with (a) different amino acid residue(s), wherein said variant(s) are capable of acting as a functional agonistic anti-CD40 monoclonal antibody as described herein above and below. Preferred are conservative substitutions in the CDR regions of the antibodies of the invention. In addition, CD40 ligand variants or orthologs comprise molecules which are at least 60%, more preferably at least 80%, 90% and most preferably at least 95% or even 99% homologous to the polypeptide as shown above in SEQ ID NO: 26 and 27, which are still capable to induce aggregation of this cognate receptor.

In order to determine whether a nucleic acid sequence has a certain degree of identity to a nucleic acid encoding agonistic anti-CD40 monoclonal antibody or CD40L orthologs, the skilled person can use means and methods well known in the art, e.g. alignments, either manually or by using computer programs such as those mentioned herein below in connection with the definition of the term “hybridization” and degrees of homology.

The term “hybridization” or “hybridizes” as used herein may relate to hybridizations under stringent or non-stringent conditions. If not further specified, the conditions are preferably non-stringent. Said hybridization conditions may be established according to conventional protocols described, e.g., in Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989), or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as 0.1×SSC, 0.1% SDS at 65° C. Non-stringent hybridization conditions for the detection of homologous or not exactly complementary sequences may be set at 6×SSC, 1% SDS at 65° C. As is well known, the length of the probe and the composition of the nucleic acid to be determined constitute further parameters of the hybridization conditions. Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility. Hybridizing nucleic acid molecules also comprise fragments of the above described molecules. Such fragments may represent nucleic acid sequences which code for an agonistic anti-CD40 monoclonal antibody or for CD40L or for a functional fragment thereof which have a length of at least 12 nucleotides, preferably at least 15, more preferably at least 18, more preferably of at least 21 nucleotides, more preferably at least 30 nucleotides, even more preferably at least 40 nucleotides and most preferably at least 60, 100, 200, 400, 600 or 1000 nucleotides. Furthermore, nucleic acid molecules which hybridize with any of the aforementioned nucleic acid molecules also include complementary fragments, derivatives and allelic variants of these molecules. Additionally, a hybridization complex refers to a complex between two nucleic acid sequences by virtue of the formation of hydrogen bonds between complementary G and C bases and between complementary A and T bases; these hydrogen bonds may be further stabilized by base stacking interactions. The two complementary nucleic acid sequences hydrogen bond in an anti-parallel configuration. A hybridization complex may be formed in solution (e.g., Cot or Rot analysis) or between one nucleic acid sequence present in solution and another nucleic acid sequence immobilized on a solid support (e.g., membranes, filters, chips, pins or glass slides to which, e.g., cells have been fixed). The terms “complementary” or “complementarity” refer to the natural binding of polynucleotides under permissive salt and temperature conditions by base-pairing. For example, the sequence “A-G-T” binds to the complementary sequence “T-C-A”. Complementarity between two single-stranded molecules may be “partial”, in which only some of the nucleic acids bind, or it may be complete when total complementarity exists between single-stranded molecules. The degree of complementarity between nucleic acid strands has significant effects on the efficiency and strength of hybridization between nucleic acid strands. This is of particular importance in amplification reactions, which depend upon binding between nucleic acids strands.

The term “hybridizing sequences” preferably refers to sequences which display a sequence identity of at least 40%, preferably at least 50%, more preferably at least 60%, even more preferably at least 70%, particularly preferred at least 80%, more particularly preferred at least 90%, even more particularly preferred at least 95% and most preferably at least 97% identity with a nucleic acid sequence as described above encoding agonistic anti-CD40 monoclonal antibody or a functional fragment thereof and being capable of acting as a functional agonistic anti-CD40 monoclonal antibody as described herein above and below and tests are described for assaying the agonistic anti-CD40 monoclonal antibody activity as described in detail below and as exemplified in the appended examples. Moreover, the term “hybridizing sequences” preferably refers to sequences encoding agonistic anti-CD40 monoclonal antibody or a functional fragment thereof having a sequence identity of at least 40%, preferably at least 50%, more preferably at least 60%, even more preferably at least 70%, particularly preferred at least 80%, more particularly preferred at least 90%, even more particularly preferred at least 95% and most preferably at least 97% identity with an amino acid sequence of the agonistic anti-CD40 monoclonal antibody sequences as described herein and being as an capable of acting as a functional agonistic anti-CD40 monoclonal antibody as described herein above and below. The same meaning can also be applied to sequences encoding CD40L or biological active fragments thereof.

In accordance with the present invention, the term “identical” or “percent identity” in the context of two or more nucleic acid or amino acid sequences, refers to two or more sequences or subsequences that are the same, or that have a specified percentage of amino acid residues or nucleotides that are the same (e.g., 60% or 65% identity, preferably, 70-95% identity, more preferably at least 95% identity with the nucleic acid sequences or with the amino acid sequences as described above and being capable of acting as a functional agonistic anti-CD40 monoclonal antibody or CD40L), when compared and aligned for maximum correspondence over a window of comparison, or over a designated region as measured using a sequence comparison algorithm as known in the art, or by manual alignment and visual inspection. Sequences having, for example, 60% to 95% or greater sequence identity are considered to be substantially identical. Such a definition also applies to the complement of a test sequence. Preferably, the described identity exists over a region that is at least about 15 to 25 amino acids or nucleotides in length, more preferably, over a region that is about 50 to 100 amino acids or nucleotides in length. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul, (1997) Nucl. Acids Res. 25:3389-3402; Altschul (1993) J. Mol. Evol. 36:290-300; Altschul (1990) J. Mol. Biol. 215:403-410). The BLASTN program for nucleic acid sequences uses as defaults a word length (W) of 11, an expectation (E) of 10, M=5, N=4, and a comparison of both strands. For amino acid sequences, the BLASTP program uses as defaults a wordlength (W) of 3, and an expectation (E) of 10. The BLOSUM62 scoring matrix (Henikoff (1989) PNAS 89:10915) uses alignments (B) of 50, expectation (E) of 10, M=5, N=4, and a comparison of both strands.

Moreover, the present invention also relates to nucleic acid molecules whose sequence is being degenerate in comparison with the sequence of an above-described hybridizing molecule. When used in accordance with the present invention the term “being degenerate as a result of the genetic code” means that due to the redundancy of the genetic code different nucleotide sequences code for the same amino acid.

In order to determine whether an amino acid residue or nucleotide residue in a nucleic acid sequence corresponds to a certain position in the amino acid sequence or nucleotide sequence of the invention, the skilled person can use means and methods well-known in the art, e.g., alignments, either manually or by using computer programs such as those mentioned further down below in connection with the definition of the term “hybridization” and degrees of homology.

For example, BLAST 2.0, which stands for Basic Local Alignment Search Tool BLAST (Altschul (1997), loc. cit.; Altschul (1993), loc. cit.; Altschul (1990), loc. cit.), can be used to search for local sequence alignments. BLAST, as discussed above, produces alignments of both nucleotide and amino acid sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cut-off score set by the user. The BLAST approach is to look for HSPs between a query sequence and a database sequence, to evaluate the statistical significance of any matches found, and to report only those matches which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identity \times \% maximum BLAST score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules. Another example for a program capable of generating sequence alignments is the CLUSTALW computer program (Thompson (1994) Nucl. Acids Res. 2:4673-4680) or FASTDB (Brutlag (1990) Comp. App. Biosci. 6:237-245), as known in the art.

The nucleic acid molecule of the present invention may be a naturally nucleic acid molecule as well as a recombinant nucleic acid molecule. The nucleic acid molecule of the invention may, therefore, be of natural origin, synthetic or semi-synthetic. It may comprise DNA, RNA as well as PNA and it may be a hybrid thereof.

It is evident to the person skilled in the art that regulatory sequences may be added to the nucleic acid molecule of the invention. For example, promoters, transcriptional enhancers and/or sequences which allow for induced expression of the polynucleotide of the invention may be employed. A suitable inducible system is for example tetracycline-regulated gene expression as described, e.g., by Gossen and Bujard (Proc. Natl. Acad. Sci. USA 89 (1992), 5547-5551) and Gossen et al. (Trends Biotech. 12 (1994), 58-62), or a dexamethasone-inducible gene expression system as described, e.g. by Crook (1989) EMBO J. 8, 513-519.

Furthermore, said nucleic acid molecule may contain, for example, thioester bonds and/or nucleotide analogues. Said modifications may be useful for the stabilization of the nucleic acid molecule against endo- and/or exonucleases in the cell. Said nucleic acid molecules may be transcribed by an appropriate vector containing a chimeric gene which allows for the transcription of said nucleic acid molecule in the cell. In this respect, it is also to be understood that the polynucleotide of the invention can be used for “gene targeting”. In a preferred embodiment said nucleic acid molecules are labeled. Methods for the detection of nucleic acids are well known in the art, e.g., Southern and Northern blotting, PCR or primer extension.

The nucleic acid molecule(s) of the invention may be a recombinantly produced chimeric nucleic acid molecule comprising any of the aforementioned nucleic acid molecules either alone or in combination. Preferably, the nucleic acid molecule of the invention is part of a vector.

The present invention therefore also relates to a vector comprising the nucleic acid molecule of the present invention. The vector of the present invention may be, e.g., a plasmid, cosmid, virus, bacteriophage or another vector used e.g. conventionally in genetic engineering, and may comprise further genes such as marker genes which allow for the selection of said vector in a suitable host cell and under suitable conditions.

Furthermore, the vector of the present invention may, in addition to the nucleic acid sequences of the invention, comprise expression control elements, allowing proper expression of the coding regions in suitable hosts. Such control elements are known to the artisan and may include a promoter, a splice cassette, translation initiation codon, translation and insertion site for introducing an insert into the vector. Preferably, the nucleic acid molecule of the invention is operatively linked to said expression control sequences allowing expression in eukaryotic or prokaryotic cells.

Control elements ensuring expression in eukaryotic and prokaryotic cells are well known to those skilled in the art. As mentioned herein above, they usually comprise regulatory sequences ensuring initiation of transcription and optionally poly-A signals ensuring termination of transcription and stabilization of the transcript. Additional regulatory elements may include transcriptional as well as translational enhancers, and/or naturally-associated or heterologous promoter regions. Possible regulatory elements permitting expression in for example mammalian host cells comprise the CMV-HSV thymidine kinase promoter, SV40, RSV-promoter (Rous Sarcoma Virus), human elongation factor 1α-promoter, the glucocorticoid-inducible MMTV-promoter (Moloney Mouse Tumor Virus), metallothionein- or tetracyclin-inducible promoters, or enhancers, like CMV enhancer or SV40-enhancer. For expression in neural cells, it is envisaged that neurofilament-, PGDF-, NSE-, PrP-, or thy-1-promoters can be employed. Said promoters are known in the art and, inter alia, described in Charron (1995), J. Biol. Chem. 270, 25739-25745. For the expression in prokaryotic cells, a multitude of promoters including, for example, the tac-lac-promoter or the trp promoter, has been described. Besides elements which are responsible for the initiation of transcription such regulatory elements may also comprise transcription termination signals, such as SV40-poly-A site or the tk-poly-A site, downstream of the polynucleotide. In this context, suitable expression vectors are known in the art such as Okayama-Berg cDNA expression vector pcDV1 (Pharmacia), pRc/CMV, pcDNA1, pcDNA3 (In-vitrogene), pVAX1 (Invitrogen), pSPORT1 (GIBCO BRL), pX (Pagano (1992) Science 255, 1144-1147), yeast two-hybrid vectors, such as pEG202 and dpJG4-5 (Gyuris (1995) Cell 75, 791-803), or prokaryotic expression vectors, such as lambda gill or pGEX (Amersham-Pharmacia). Beside the nucleic acid molecules of the present invention, the vector may further comprise nucleic acid sequences encoding for secretion signals. Such sequences are well known to the person skilled in the art. Furthermore, depending on the expression system used leader sequences capable of directing the peptides of the invention to a cellular compartment may be added to the coding sequence of the nucleic acid molecules of the invention and are well known in the art. The leader sequence(s) is (are) assembled in appropriate phase with translation, initiation and termination sequences, and preferably, a leader sequence capable of directing secretion of translated protein, or a protein thereof, into the periplasmic space or extracellular medium. Optionally, the heterologous sequence can encode a fusionprotein including an C- or N-terminal identification peptide imparting desired characteristics, e.g., stabilization or simplified purification of expressed recombinant product. Once the vector has been incorporated into the appropriate host, the host is maintained under conditions suitable for high level expression of the nucleotide sequences, and, as desired, the collection and purification of the antibody molecules or recombinant proteins or fragments thereof of the invention may follow.

The present invention also relates to a host cell transfected or transformed with the vector of the invention or a non-human host carrying the vector of the present invention, i.e. to a host cell or host which is genetically modified with a nucleic acid molecule according to the invention or with a vector comprising such a nucleic acid molecule. The term “genetically modified” means that the host cell or host comprises in addition to its natural genome a nucleic acid molecule or vector according to the invention which was introduced into the cell or host or into one of its predecessors/parents. The nucleic acid molecule or vector may be present in the genetically modified host cell or host either as an independent molecule outside the genome, preferably as a molecule which is capable of replication, or it may be stably integrated into the genome of the host cell or host.

The host cell of the present invention may be any prokaryotic or eukaryotic cell. Suitable prokaryotic cells are those generally used for cloning like E. coli or Bacillus subtilis. Furthermore, eukaryotic cells comprise, for example, fungal or animal cells. Examples for suitable fungal cells are yeast cells, preferably those of the genus Saccharomyces and most preferably those of the species Saccharomyces cerevisiae. Suitable animal cells are, for instance, insect cells, vertebrate cells, preferably mammalian cells, such as e.g. HEK293, NSO, CHO, MDCK, U2-OSHela, NIH3T3, MOLT-4, Jurkat, PC-12, PC-3, IMR, NT2N, Sk-n-sh, CaSki, C33A. These host cells, e.g. CHO-cells, may provide posts-translational modifications to the antibody molecules of the invention, including leader peptide removal, folding and assembly of H and C chains, glycosylation of the molecule at correct sides and secretion of the functional molecule. Further suitable cell lines known in the art are obtainable from cell line depositories, like the American Type Culture Collection (ATCC). In accordance with the present invention, it is furthermore envisaged that primary cells/cell cultures may function as host cells. Said cells are in particular derived from insects (like insects of the species Drosophila or Blatta) or mammals (like human, swine, mouse or rat). The above mentioned primary cells are well known in the art.

In a more preferred embodiment the host cell which is transformed with the vector of the invention is a COS-7 cell line or a cell (line) derived therefrom. However, also a CHO-cell comprising the nucleic acid molecule of the present invention may be particularly useful as host. Such cells may provide for correct secondary modifications on the expressed molecules, i.e. the antibody molecules or CD40L of the present invention. These modifications comprise, inter alia, glycosylations and phosphorylations.

Hosts may be non-human mammals, most preferably mice, rats, sheep, calves, dogs, monkeys or apes. Furthermore, the hosts of the present invention may be partially useful in producing the antibody molecules (or fragments thereof) of the invention. It is envisaged that said antibody molecules or CD40L (or fragments thereof) be isolated from said host. It is, inter alia, envisaged that the nucleic acid molecules and or vectors described herein are incorporated in sequences for transgenic expression. The introduction of the inventive nucleic acid molecules as transgenes into non-human hosts and their subsequent expression may be employed for the production of the inventive antibodies. For example, the expression of such (a) transgene(s) in the milk of the transgenic animal provide for means to obtain the inventive antibody molecules in quantitative amounts; see inter alia, U.S. Pat. No. 5,741,957, U.S. Pat. No. 5,304,489 or U.S. Pat. No. 5,849,992. Useful transgenes in this respect comprise the nucleic acid molecules of the invention, for example, coding sequences for the light and heavy chains of the antibody molecules described herein, operatively linked with promoter and/or enhancer structures from a mammary gland specific gene, like casein or beta-lactoglobulin.

The invention also provides for a method for the preparation of an antibody molecule or a recombinant protein of the invention comprising culturing the host cell described herein above under conditions that allow synthesis of said antibody molecule or CD40L and recovering said antibody molecule or CD40L from said culture.

The invention also relates to a vaccine comprising the combination of an antibody molecule or CD40L of the invention or produced by the method described herein above, a nucleic acid molecule encoding for the antibody molecule or CD40L of the invention, a vector comprising said nucleic acid molecule or a host-cell as defined herein above and optionally, further molecules, either alone or in combination, in particular in combination with the inactivated or attenuated bacteria as described above. The term “vaccine” as used herein is known to the person skilled in the art and relates in general to a biological preparation that improves immunity to a particular disease. A vaccine typically contains an agent that resembles a disease-causing microorganism, and is often made from weakened or killed forms of the microbe or its toxins. The agent stimulates the body's immune system to recognize the agent as foreign, destroy it, and “recognize” it, so that the immune system can more easily recognize and destroy any of these microorganisms that it later encounters. The term “vaccine” as employed herein comprises at least one compound of the invention. Preferably, such a vaccine is a pharmaceutical composition.

Accordingly, in a preferred embodiment, the invention provides a vaccine comprising the combination of an agonistic anti-CD40 monoclonal antibody or CD40L or a nucleic acid molecule encoding for the anti-CD40 antibody or a nucleic acid molecule encoding for CD40L, and inactivated or attenuated bacteria selected from the group consisting of Staphylococcus, Streptococcus, Listeria or Escherichia for use in the treatment and/or prevention of mastitis. In a more preferred embodiment, the vaccine comprises the above mentioned combination, wherein the agonistic anti-CD40 monoclonal antibody comprises a) an immunoglobulin heavy chain variable domain (VH) which comprises the hypervariable regions CDR1, CDR2 and CDR3, said CDR1 having the amino acid sequence SEQ ID N01: SYAMS, said CDR2 having the amino acid sequence SEQ ID NO:2: SIGSGGGTYYPDSVKD, and said CDR3 having the amino acid sequence SEQ ID NO:3: AYYRNHRGSVMDY; and b) an immunoglobulin light chain variable domain (VL) which comprises the hypervariable regions CDR1′, CDR2′ and CDR3′, said CDR1′ having the amino acid sequence SEQ ID NO4: KASQTVDYDGDSYMN, said CDR2′ having the amino acid sequence SEQ ID NO5: SASNLES, and said CDR3′ having the amino acid sequence SEQ ID NO6: QQSTEDPPT; for use in the treatment and/or prevention of mastitis; or variants thereof capable of inducing CD40 receptor aggregation. In yet another preferred embodiment, the vaccine comprises the above mentioned combination, wherein the CD40L comprises the amino acids 47 to 261 or 46 to 261, of the SEQ ID NO26 or SEQ ID NO27, or a biological active fragment thereof.

In yet another more preferred embodiment, the vaccine comprises the above mentioned combination, wherein the agonistic anti-CD40 monoclonal antibody is an anti-bovine CD40 antibody. In a more preferred embodiment, the vaccine of the invention comprises the combination mentioned above, wherein the antibody is produced by the hybridoma cells deposited on Apr. 2, 2010 at the International Depositary Authority of the Belgian Coordinated Collections of Microorganisms (BCCM/LMBP) Collection, Department of Molecular Biology, Ghent University, Technologiepark 927, B-9052 Gent-Zwijnaarde, Belgium, in accordance with the Budapest Treaty of 28 Apr. 1977 on the International Recognition of the Deposit of Microorganisms for the Purposes of Patent Procedure under No: LMBP 7218CB [i.e. clone E1]. Said hybridoma cells (i.e. the clone E1) are issued from the fusion of spleen lymphocytes (isolated from mice immunized with cells transfected with a plasmid coding the bovine CD40 receptor) with myeloma cells which lacks the hypoxanthine-guanine phosphoribosyltransferase. After a selection with hypoxanthine-aminopterin-thymidine, single cells per well culture (96 wells plate) were assessed for IgG production by ELISA screening and after for specificity to bovine CD40 receptor. The clone E1 is one of the clones issued from this experiment.

Furthermore, in another preferred embodiment, in the vaccine of the present invention the Staphylococcus is Staphylococcus aureus or Staphylococcus agalactiae, the Streptococcus is Streptococcus uberis, the Escherichia is Eschericha coli and the Listeria is Listeria monocytogenes.

In a further aspect, the vaccine comprises the above mentioned combination for use in the treatment of mastitis in dairy cattle, sheep or goats.

Consequently, without being bound by theory, the present invention provides for the above described vaccine that has medical implications, i.e. the ability, as exemplified in the appended examples, to induce a strong CTL response, wherein said strong CTL response is capable of protecting, treating or preventing mastitis. Consequently, the induction of the CTL response is expected to have medical implications, i.e., e.g., to reduce the negative impact of infections like intracellular bovine infections caused by intracellular pathogens, i.e., e.g. mastitis. Preferably, said intracellular pathogen is Staphylococcus aureus (S. aureus). Staphylococcus aureus, a common gram-positive bacterium, is the most prevalent infectious agent that causes subclinical mastitis. This high prevalence is partly explained by the fact that staphylococcal mastitis remains difficult to treat and/or control efficiently. This is notably related to the ability of S. aureus to invade and survive within host phagocytes and mammary epithelial cells. Indeed, intracellular invasion provides protection from the humoral immune response and several classes of antibiotics. To reduce the negative impact of S. aureus infections, the present invention has surprisingly found that the agonistic anti-CD40 monoclonal antibody or CD40 ligand (CD40L) or a vector coding for the anti-CD40 antibody or a vector coding for the CD40L displays agonistic properties, and, as exemplified in the appended examples, avoids intracellular persistence of the pathogen. Accordingly, to avoid this intracellular persistence, the vaccine strategy of the present invention is based on the induction of a strong cytotoxic T lymphocyte (CTL) response. To achieve this kind of immune response in vivo, the above mentioned agonistic anti-CD40 monoclonal antibodies (mAbs) or CD40 ligand (CD40L) or a vector coding for the anti-CD40 antibody or a vector coding for the CD40L of the vaccine are used to polarize the immune response towards a CTL response against S. aureus. As demonstrated in the appended examples, immunization of mice with heat-killed S. aureus (HKSA) together with agonistic anti-CD40 mAbs elicits strong CTL responses capable of protecting mice from subsequent staphylococcal mastitis. As already mentioned above, said intracellular pathogen is preferably Staphylococcus aureus (S. aureus). However, it is also envisaged to use the vaccine strategy of the present invention for other intracellular infections, preferably bovine infections, caused by other bacteria that cause mastitis as outlined above as well as other intracellular pathogens, i.e. viruses or parasites.

The vaccine may be in solid, liquid or gaseous form and may be, inter alia, in a form of (a) powder(s), (a) tablet(s), (a) solution(s) or (an) aerosol(s). Said composition may comprise at least two, preferably three, more preferably four, most preferably five antibody molecules of the invention or nucleic acid molecules encoding said antibody molecules or CD40L proteins or nucleic acid molecules encoding the CD40L proteins.

It is preferred that said vaccine optionally comprises a pharmaceutically acceptable carrier and/or diluent. The herein disclosed vaccine may be partially useful for the treatment of mastitis, preferably in dairy cattle, sheep or goats.

Examples of suitable pharmaceutical carriers, excipients and/or diluents are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. It is particularly preferred that said administration is carried out by injection and/or delivery, e.g., subcutaneously, intramammary and/or intramuscularly. The compositions of the invention may also be administered directly to the target site. The dosage regimen will be determined by the attending physician and clinical factors. As is well known in the medical arts, dosages for any one patient depends upon many factors, including the patient's or subject's size, body surface area, age, the particular compound to be administered, sex, time and route of administration, general health, and other drugs being administered concurrently. Proteinaceous pharmaceutically active matter may be present in amounts between 1 μg and 10 mg/kg body weight per dose; however, doses below or above this exemplary range are envisioned, especially considering the aforementioned factors. If the regimen is a continuous infusion, it should also be in the range of 1 μg to 10 mg units per kilogram of body weight per minute.

Progress can be monitored by periodic assessment. The vaccines of the invention may be administered locally or systemically. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like.

Preservatives and other additives may also be present such as, for example, antimicrobials, anti-oxidants, chelating agents, and inert gases and the like. Furthermore, the vaccine of the invention may comprise further agents depending on the intended use of the vaccine. Said agents may be, e.g., Tween, EDTA, Citrate, Sucrose as well as other agents being suitable for the intended use of the vaccine that are well-known to the person skilled in the art.

In yet another embodiment, the present invention provides for a kit comprising at least one antibody molecule, at least one nucleic acid molecule encoding the antibody, at least one CD40L protein, at least one nucleic acid molecule encoding the CD40L protein, at least one vector, at least one host cell of the invention and/or at least one inactivated or attenuated bacteria preparation of the invention. Advantageously, the kit of the present invention further comprises, optionally (a) buffer(s), storage solutions and/or remaining reagents or materials required for the conduct of medical, scientific or diagnostic assays and purposes. Furthermore, parts of the kit of the invention can be packaged individually in vials or bottles or in combination in containers or multicontainer units.

The kit of the present invention may be advantageously used, inter alia, for carrying out the method of the invention and could be employed in a variety of applications referred herein, e.g., as diagnostic kits, as research tools or medical tools. Additionally, the kit of the invention may contain means for detection suitable for scientific, medical and/or diagnostic purposes. The manufacture of the kits follows preferably standard procedures which are known to the person skilled in the art.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in this example.

### Example I

### CD40 Triggering Induces Strong Cytotoxic T Lymphocyte Responses to Heat-Killed Staphylococcus aureus: a New Vaccine Strategy for Staphylococcal Mastitis

### Materials and Methods as Employed in the Following

### 1. Mice

Wild-type C57BL/6 and BALB/c mice were purchased from Harland Nederland. OT-II mice (C57BL/6 background) transgenic for αβ-TCR reactive with the I-A^{b}-restricted 323-339 peptide of ovalbumin (OVA), and OT-I mice (C57BL/6 background) transgenic for αβ-TCR reactive with the H-2K^{b}-restricted 257-264 peptide of OVA, were from the Jackson Laboratory. All mice were housed in our specific pathogen free facility and used at 6-10 week of age, except lactating mice that were used at 14-20 week of age. All experiments were conducted with Institutional Animal Care and Use Committee approval.

### 2. Bacteria

S. aureus Newbould 305 (American Type Culture Collection 29740), a mastitis isolate, was the pathogen used. Prior to each experiment, a single colony from a Nutrient (Difco Laboratories) agar plate was inoculated into 4 ml of Nutrient Broth and grown at 37° C. with vigorous shaking for 7 h. From this 4-ml culture, 100 μl were transferred into 10 ml of Nutrient Broth and incubated overnight (16 h) at 37° C. with vigorous shaking. The overnight culture was centrifuged, and the pellet was resuspended in PBS at a density of 10¹⁰ CFU/ml. Dilutions of this stock suspension were used in the experiments described below.

For preparation of HKSA, bacteria were incubated at 60° C. for 75 min. Effective bacterial killing was confirmed by incubating a 100-μl aliquot in Nutrient Broth overnight at 37° C.

### 3. Antibodies and Reagents

Agonistic anti-CD40 mAbs (hereafter referred to as αCD40) were from R&D Systems (1C10, rat IgG). APC-conjugated anti-TCR Vα2 (B20.1), and Pacific Blue®-conjugated anti-CD4 (GK1.5) and anti-CD8α (KT15) were from eBioscience. FITC-conjugated anti-CD8α (53-6.7) were from BD Biosciences. Anti-Fcg II/III (2.4G2) antibodies were produced in house.

The OTI (chicken OVA peptide 257-264 SIINFEKL) peptide was purchased from Neosystem. Carboxyfluorescein succinimidyl ester (CFSE) was from Molecular Probes Invitrogen. Phorbol myristate acetate (PMA), ionomycin, and OVA grade V were purchased from Sigma.

### 4. Flow Cytometry

Staining reactions were performed at 4° C. Cells were incubated with 2.4G2 Fc receptor Abs to reduce non specific binding. Stained cells were analyzed on a FACSCanto™ II (BD Biosciences).

### 5. Immunization Protocols

On day 0, mice were injected subcutaneously (s.c.) between the L4 and R4 abdominal mammary glands with either 10 μg OVA or 5×10⁸ HKSA. Immediately afterward, they were given 25μαCD40 intraperitoneally (i.p.). Control mice were administered with either OVA alone, HKSA alone, αCD40 alone, or PBS.

### 6. Restimulation of Mammary Lymph Nodes

Five days after immunization (day 5), mammary lymph node (MLN) cells were isolated and cultured in Click's medium supplemented with 0.5% heat-inactivated mouse serum and additives (2 mM L-glutamine, 1 mM sodium pyruvate, 0.1 mM non essential amino acids, 50 μM β-mercaptoethanol, 50 μg/ml streptomycin and 50 IU/ml penicillin). MLN cells were left untreated or were restimulated with either 50 μg/ml OVA or 1×10⁵ HKSA. The proliferation of MLN cells was measured as ³H-thymidine incorporation during the last 16 h of a 3-day culture. Culture supernatants were assayed for interferon (IFN)-γ and interleukin (IL)-4 by ELISA (Biosource).

### 7. In Vivo Proliferation of OVA-Specific CD4⁺ and CD8⁺ T Cells

On day −1, mice received an intravenous (i.v.) injection of CFSE-labeled OT-I or OT-II cells (equivalent of 10×10⁶ transgenic T cells). On day 0, mice were immunized with OVA administered together with αCD40. Control mice received either OVA alone, αCD40 alone, or PBS. On day 4, T cell responses in MLNs were analyzed by observing CFSE division profiles of live TCR Vα2⁺ CD4⁺ (OT-II) or CD8⁺ (OT-I) T cells.

CFSE labeling: splenic and lymph node cells from OT-I or OT-II mice (5×10⁷ cells/ml) were incubated with CFSE (0.5 μM in PBS) for 10 min at 37° C. Cells were washed in PBS containing 10% fetal calf serum and then in PBS, and injected i.v.

### 8. Intracellular IFN-γ Staining

Mice were immunized with OVA and αCD40 at day 0. On day 5, MLN cells were collected and treated with PMA (10 ng/ml) and ionomycin (250 ng/ml) for 6 h and incubated with 1 μg/ml Brefeldin A (GolgiPlug, BD Biosciences) for the last 4 h. Cells were stained for either CD4 or CD8. Cells were then fixed and permeabilized with BD Cytofix/Cytoperm™ Fixation/Permeabilization Kit (BD Biosciences), and stained intracellularly with an APC-conjugated anti IFN-γ Ab (XMG1.2; eBioscience).

Alternatively, mice were immunized with HKSA and αCD40. In this case, MLN cells were stimulated with 1×10⁵ HKSA/ml for 20 h and incubated with Brefeldin A for the last 4 h.

### 9. In Vivo CTL Assays

Splenocytes and lymph node cells from naive C57BL/6 mice were pulsed or not with 5 μg/ml OT-I peptide and labeled with CFSE. Peptide-pulsed cells were labeled with CFSE at a final concentration of 5 μM and unpulsed cells were labeled at a 10-fold lower concentration by incubation for 10 min at 37° C. The two cell populations were mixed at a 1:1 ratio and 5×10⁷ cells were injected i.v. into recipient mice. Recipient mice were immunized with OVA and αCD40 5 days before i.v. injection of CFSE-labeled targets. MLNs were excised 16 h later after the injection of unpulsed/pulsed cells and single cell suspensions were analyzed for detection and quantification of CFSE-labeled cells. The percentage of antigen-specific lysis in vivo was calculated as follows: (1−number of CFSE^{hi} cells/number of CFSE^{lo} cells)×100.

### 10. Effects of HKSA Immunization in a Mouse Model of Staphylococcal Mastitis

BALB/c lactating mice were immunized with HKSA and αCD40 seven days after birth of the offspring. Control mice were injected with either HKSA alone, αCD40 alone, or PBS. The pups were removed at day 14, 1 h before bacterial inoculation of mammary glands. Lactating mice were anesthetized by i.p. injection of xylazine (200 μg/mouse) and ketamine (2 mg/mouse). Lactiferous duct of both the L4 (on the left) and R4 (on the right) abdominal mammary glands were exposed by a cut under a binocular and 100 μl bacterial suspension (10² CFU/gland) was injected through the orifice. These large glands constitute the fourth pair found from the head to the tail. On day 15, mice were killed by cervical dislocation and mammary glands were aseptically harvested, weighted and homogenized in a final volume of 2 ml of PBS. The two infected mammary glands, which are structurally separated, were considered as individual samples throughout the experiments. The bacterial counts (CFU) were determined by plating logarithmic dilutions of the samples on mannitol salt agar plates in quadriplicate and number of S. aureus CFU were counted 16 h later. Raw bacterial CFU counts were transformed in base-10 logarithm values and data represented by medians.

### 11. Depletion of CM⁺ T Cells

For depletion of CD8⁺ T cells, mice were treated i.p. 3 days before and at the time of infection with 300 μg anti-CD8 mAbs (clone YTS169; Abeam).

### 12. Statistical Analysis

Data are presented as means±standard deviations (SDs). The differences between mean values were estimated using an analysis of variance test followed by a Fisher's protected least standard deviation test. Alternatively, Mann-Whitney U-tests were used (in experiments involving the mouse model of staphylococcal mastitis). A value of P<0.05 was considered significant. All the experiments were repeated at least three times. n≧6 in each mice group.

### Results

### 1: Induction of OVA-Specific CTL Responses in the Mammary Gland by Co-Administration of OVA and αCD40

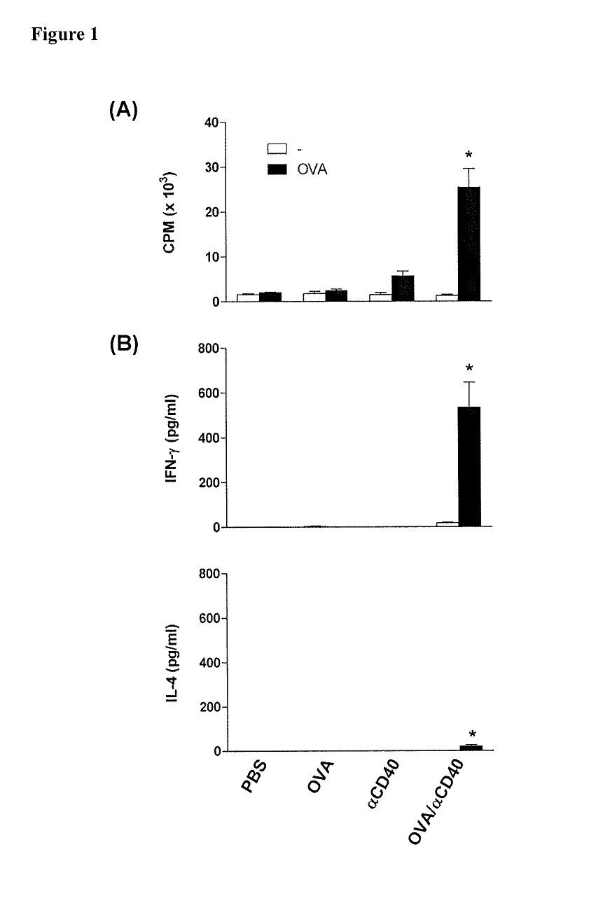

Previous studies have shown that stimulation of host APCs by αCD40 markedly enhances CTL responses to tumor cells and some intracellular pathogens such as Listeria monocytogenes and Leishmania major. To test whether αCD40 are also able to promote CD8⁺ T cell-mediated immunity against intramammary inoculated antigens, naive C57BL/6 mice were injected s.c. between the L4 and R4 abdominal mammary glands with 10 μg OVA and immediately afterward were given 25 μg αCD40 i.p. Control mice received either OVA alone, αCD40 alone, or PBS. Five days later, MLN cells were collected and restimulated in vitro with OVA. MLN cells from mice that were injected with αCD40 and OVA (hereafter referred to as OVA/αCD40 mice) exhibited a significant proliferative response in the presence of OVA, whereas no or little proliferative response was observed in MLN cells from control mice (FIG. 1a). To better characterize the immune response induced by co-administration of αCD40 and OVA, the concentrations of IFN-γ and IL-4 in the supernatant of OVA-restimulated MLN cells were assessed by ELISA. Neither cytokine was detected in the supernatant of MLN cells from control mice (FIG. 1b). In contrast, immunization with OVA and αCD40 was associated with very high levels of IFN-γ production (FIG. 1b). MLN cells from αCD40/OVA mice also secreted IL-4, but at low levels compared with IFN-γ (FIG. 1b).

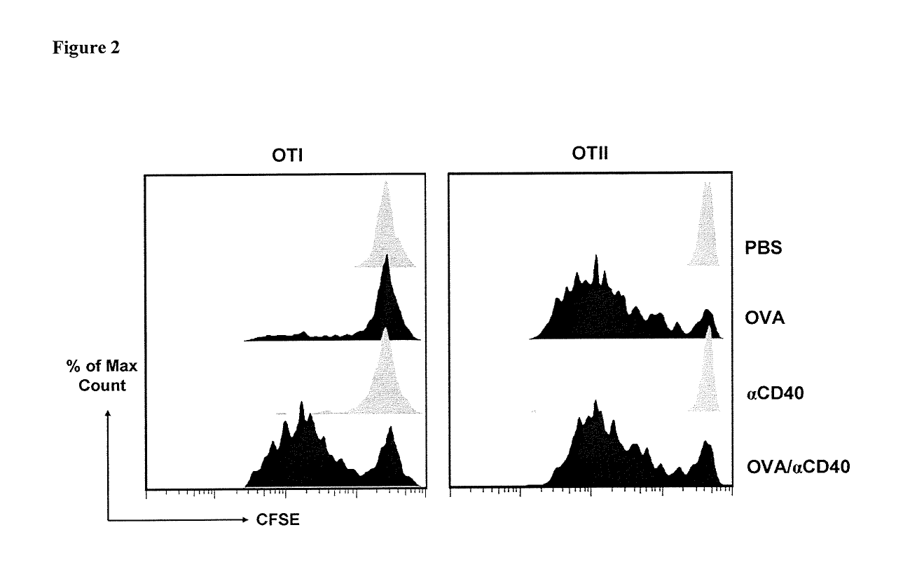

CD4⁺ and CD8⁺ T cells can both produce significant amounts of IFN-γ. To determine which T cell population was induced in OVA/αCD40 mice, naive C57BL/6 mice were transferred with CFSE-labelled OT-I or OT-II transgenic T cells. Twenty-four hours later, transferred mice were injected with either PBS, OVA, αCD40, or OVA/αCD40. Three days after immunization, MLNs were collected and analyzed by flow cytometry for the proliferation of CFSE-labelled T cells. Immunization with OVA and αCD40 induced strong proliferation of both OVA-specific CD4′ (OT-II) and CD8⁺ (OT-I) T cells, whereas no division was observed in mice injected with PBS or αCD40 alone (FIG. 2). Of note, immunization with OVA alone induced proliferation of OT-II but not OT-I T cells (FIG. 2).

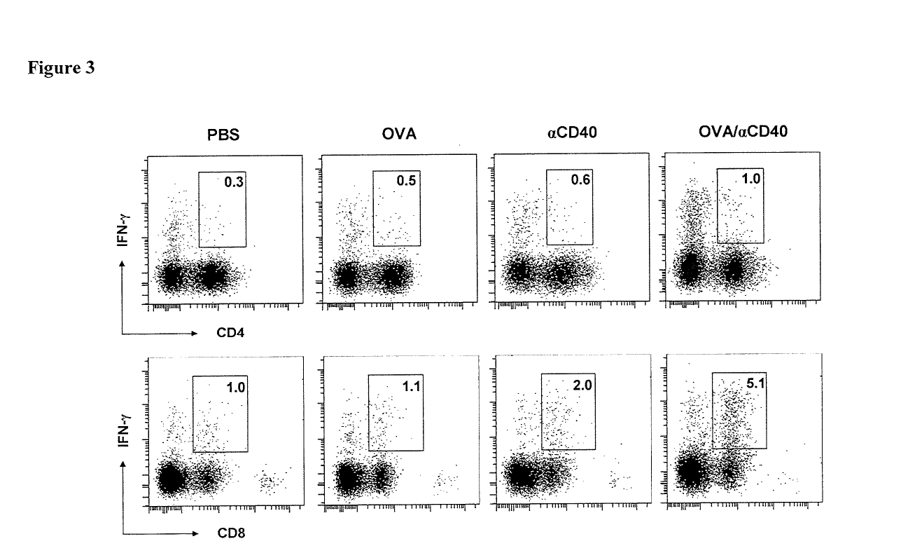

To clearly identify the cellular source of IFN-γ, we next performed intracellular staining experiments. Mice were injected with either PBS, OVA, αCD40, or OVA/αCD40. Five days later, MLN cells were collected and stimulated with PMA and ionomycin for 6 hours. CD4 and CD8 cells were then stained intracellularly for IFN-γ. Although some IFN-γ-secreting CD4⁺ T cells could be found in the MLNs from OVA/αCD40 mice, the major source of IFN-γ in these mice was the CD8⁺ T cell population (FIG. 3). CD4⁺ and CD8⁺ T cells from control mice produced only small amounts of IFN-γ (FIG. 3).

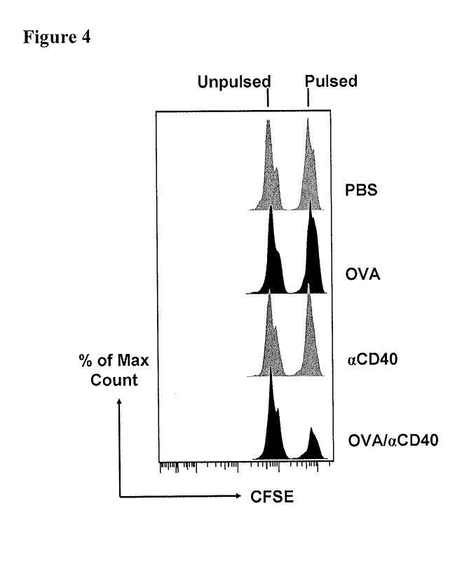

To further characterize the functional properties of OVA-specific CD8⁺ T cells generated by immunization with OVA/αCD40, an in vivo cytotoxicity assay was performed as described by Aichele et al. [8]. In brief, splenocytes and lymph node cells, pulsed or not with the OT-I peptide, were differentially labelled with CFSE (high or low, respectively) and transferred into primed recipients. Recipients were injected with either PBS, OVA, αCD40, or OVA/αCD40 5 days before injection of CFSE-labelled cells. No lysis of OT-I peptide-pulsed CFSE^{high} splenocytes was observed in recipient mice that were injected with PBS, OVA, or αCD40 (FIG. 4). By contrast, the OT-I peptide-pulsed CFSE^{high} cell population was greatly reduced in numbers, when compared with the unpulsed CFSE^{lo} population, in the MLNs of mice primed with OVA/αCD40 (FIG. 4).

Altogether, these results show that immunization with OVA/αCD40 induces the development, in the MLNs, of IFN-γ-secreting CD8⁺ cells exhibiting a high CTL activity.

### 2: HKSA/αCD40 Immunization Primes IFN-γ-Producing CD8⁺ T Cells in the MLNs

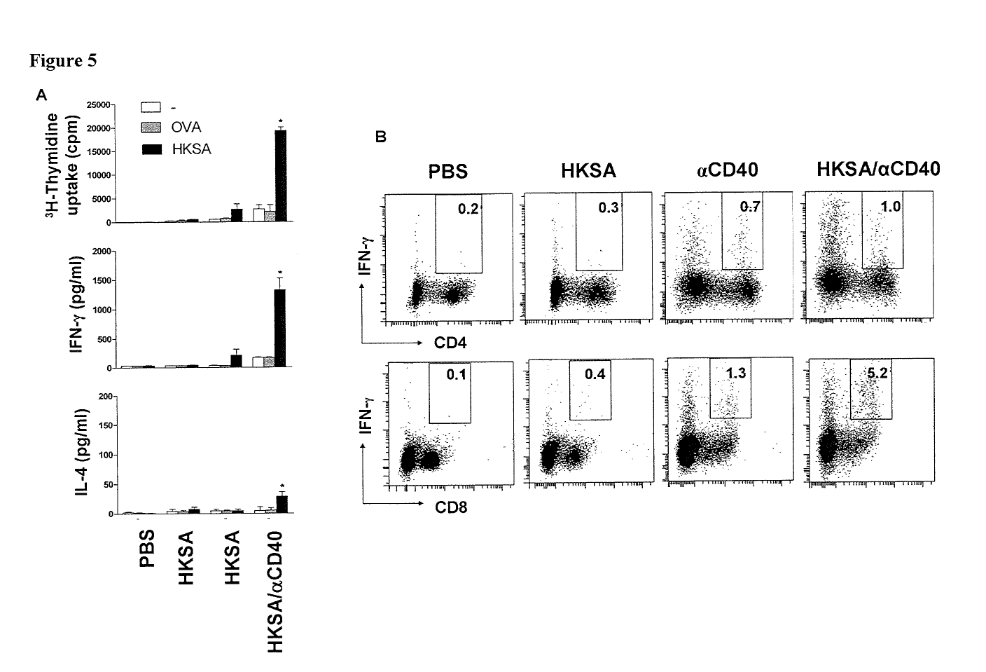

In the first part of this work, we have shown, by using a soluble model antigen (OVA), that it is possible to trigger strong CTL responses in the MLNs. We next tried to determine whether it was also feasible to induce CD8⁺ T cells directed to HKSA in these lymph nodes. HKSA injection alone is known to elicit little or no detectable immune response [9]. Our hypothesis was that CD40 stimulation of mammary APCs could convert this weakly immunogenic antigen to an immunogenic vaccine. The experiments described in FIGS. 1 and 3 were therefore reiterated with HKSA instead of OVA. MLN cells from mice immunized with HKSA/αCD40 displayed strongly increased proliferation and IFN-γ production upon in vitro stimulation with HKSA relative to MLN cells from mice that were injected with either PBS, HKSA alone, or αCD40 alone (FIG. 5a). Of note, MLN cells from HKSA/αCD40 also produced low amounts of IL-4 (FIG. 5a). These effects were antigen specific. Indeed, MLNs from mice immunized with HKSA/αCD40 did not proliferate and did not produce any cytokine when they were stimulated in vitro with OVA rather than with HKSA (FIG. 5a). As described for OVA, the major source of IFN-γ was the CD8⁺ T cell population (FIG. 5b). These data confirm that immunization with HKSA alone is not sufficient to trigger immune responses directed against this bacterium and show that immunization of mice with HKSA together with αCD40 treatment favours the development, in the MLNs, of IFN-γ-secreting CD8⁺ T cells specifically directed to HKSA.

### 3: Induction of HKSA-Specific CD8⁺ T Cells by Injection of HKSA/αCD40 Protects Mice Against Staphylococcal Mastitis

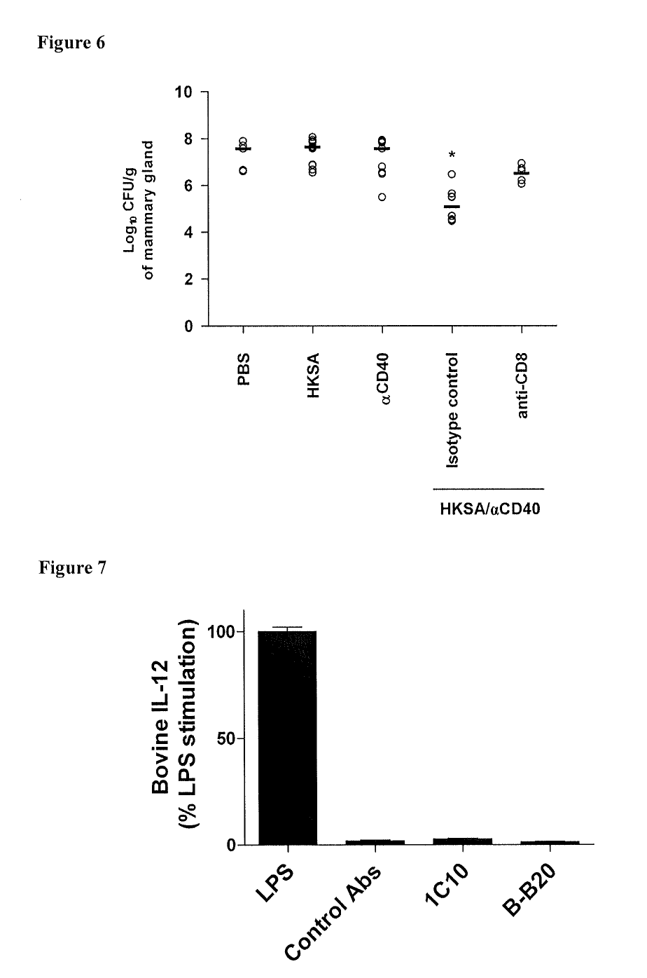

S. aureus may invade and survive within host mammary cells [2]. This suggests that induction of efficient CTL responses to S. aureus could help in controlling S. aureus mastitis. In the last part of this work, we have tested this hypothesis in a murine model of staphylococcal mastitis. BALB/c mice were immunized with HKSA/αCD40 7 days prior to mammary gland infection by S. aureus. Control mice were injected with either PBS, HKSA alone, or αCD40 alone. Twelve hours after challenge, mammary glands were harvested and homogenized. Homogenates were serially diluted and plated for CFU determination. High bacterial loads were detected in control mice treated with PBS (median recovery value: 7.58 log 10 CFU/g). By contrast, the numbers of S. aureus CFU recovered from the mammary glands of mice immunized with HKSA/αCD40 were significantly lower (5.09 log 10 CFU/g) than in control mice, an effect not observed in HKSA- and αCD40-treated mice (respectively 7.65 and 7.58 log 10 CFU/g) (FIG. 6). The contribution of HKSA-specific CD8⁺ T cells to the protection conferred by HKSA/αCD40 vaccination has been tested by depleting CD8⁺ T cells from HKSA/αCD40-vaccinated mice immediately before intramammary challenge with S. aureus. FIG. 6 shows that depletion of CD8⁺ T cells markedly reduced the efficacy of HKSA/αCD40 immunization. Indeed, the numbers of S. aureus CFU in the mammary glands of HKSA/αCD40 mice that received depleting antibodies were significantly higher than in control HKSA/αCD40 mice (6.63 vs 5.09 log 10 CFU/g). Taken together, these results demonstrate that HKSA/αCD40 vaccination provides a significant protection against intramammary S. aureus challenge and that this protection requires cytotoxic CD8⁺ T cells.

### Conclusion

Vaccination is generally considered to be one of the most effective strategies to prevent or treat diseases. Nevertheless, a vaccine that is highly effective against bovine S. aureus mastitis is not available yet [2]. The limited success of previous vaccine trials to prevent S. aureus mastitis may be due to the internalization and intracellular survival of S. aureus [3, 4]. Eradication of intracellular bacteria may only be achieved if an appropriate CTL response is induced. Our results indicate that CD40 triggering may induce, in MLNs, a strong CTL response against intramammary inoculated antigens. Most importantly, we demonstrate that immunization with HKSA combined with αCD40 protects mice against experimental S. aureus mastitis.

In this study, we used OVA as a model antigen to prove that CD40 activation is sufficient to induce strong and antigen-specific CTL responses in MLNs. It has already been shown in other models that immunisation with OVA and αCD40 may induce efficient OVA-specific CD8⁺ T cells. With regard to the latter, it is known that CD40 activation in DCs leads to heightened ability to present antigens and to produce cytokines and chemokines. Moreover, upon CD40 activation, DCs are capable of capturing exogenous Ag for the generation of MHC class I/peptide complexes by cross presentation and of polarizing the immune response towards a CTL response.

We used a whole heat-killed antigen, HKSA, as vaccine antigen. Immunization of mice with HKSA injected alone induced poor immunity. This low immunogenicity of HKSA has already been described. Some adjuvants have been shown to convert low immune responses into strong CTL responses. The combination of an exogenous antigen with αCD40 and Toll-like receptor (TLR) agonists is currently the most effective published strategy to induce specific CD8⁺ T cell responses in mice [7]. TLRs are pattern recognition receptors that can recognize pathogens via pathogen-specific molecular patters (PAMPs). Because of their ability to stimulate innate and adaptive immune responses, TLR agonists are used as vaccine adjuvants to improved protective immunity in mice models. Interestingly, TLR2 and TLR4 receptors recognize and are activated by HKSA. These observations suggest that two concomitant mechanisms favour the development of CTL responses in our model; the engagement of CD40 in APCs by αCD40 and the concomitant activation of TLR2 and TLR4 by HKSA itself.

We have tested our vaccinal strategy in a mouse model of staphylococcal mastitis because it has been reported that S. aureus inoculation in the mouse mammary gland induces similar polymorphonuclear infiltration, tissue damage, and S. aureus-host cell interactions to those observed in the bovine mammary gland. By using this model, we have shown that immunization of mice with HKSA/αCD40 protects against experimental S. aureus mastitis. The vaccine efficacy was lost following depletion of CD8⁺ T cells, demonstrating the predominant contribution of CTL responses in the protection. This observation clearly indicate that eradication of intracellular S. aureus by inducing strong CTL responses is a promising approach for the design of vaccine protecting lactating cows from S. aureus chronic mastitis. Our study also provides the first demonstration of the potential of αCD40 as an adjuvant for vaccination against a S. aureus infection. This approach could be used to prevent staphylococcal mastitis in cows but could also be extended to any chronic or fatal disease involving S. aureus.

We used the S. aureus Newbould 305 strain, a mastitis isolate, as a model pathogen. The significant antigen variation among the different strains of S. aureus responsible for mastitis has to be considered as an important factor in the development of a protecting vaccine [2]. In our vaccine strategy, this problem could be easily avoided by integrating various HKSA strains in the same vaccine.

In conclusion, we show in a mouse model that combining CD40 activation with HKSA immunization is an efficient approach for inducing HKSA-specific CTL responses in MLNs and for vaccinating against staphylococcal mastitis. Because there is no bovine αCD40 available and because neither murine nor human αCD40 are efficient in the bovine species (our unpublished results), the development of bovine αCD40 is required to evaluate if the promising vaccine strategy we describe is able to protect lactating cows against S. aureus mastitis.

### Example II

### Generation of an Agonistic Anti-Bovine CD40 Monoclonal Antibody that Induces Maturation of Dendritic Cells In Vitro and Cytotoxic T Lymphocytes Responses in Vivo

### Materials and Methods as Employed in the Following

### 1. Animals

Wild-type BALB/c mice were purchased from Harlan Nederland. All mice were housed in our specific pathogen free facility and used at 6-10 week of age. Eighteen healthy Holstein heifers were selected from neighbouring farms and housed in our large animal facility. All experiments were conducted with Institutional Animal Care and Use Committee approval.

### 2. Antibodies and Reagents

Agonistic rat anti-murine CD40 antibodies (clone 1C10) were purchased from R&D. Agonistic anti-human CD40 antibodies (clone B-B20) were from Abcam. FITC-conjugated anti-FLAG antibodies (clone M2) were from Sigma-Aldrich. Alexa-647 conjugated anti-bovine-interferon-γ (IFN-γ), FITC-conjugated anti-bovine-CD4, RPE-conjugated anti-bovine-CD8, anti-bovine-IL12 (clone CC301 and CC326), and anti-bovine-IL4 antibodies, as well as recombinant bovine IL-4 and granulocyte macrophage colony-stimulating factor (GM-CSF) were purchased from Serotec.

### 3. Cells and Transfection

NIH3T3, COS-7 and P3X63Ag8.653 cells were obtained from the American Type Culture Collection (Rockville, Md.). The cells were maintained in RPMI 1640, supplemented with 2 mM L-glutamine, 1% MEM non essential amino acids and 10% Fetal bovine serum. For transient transfection, NIH-3T3 (1.8×10⁶ cells/dish) and COS-7 cells (1.4×10⁶ cells/dish) were seeded in tissue culture dishes 24 h prior to transfection. Plasmids were transfected using TransFectin reagents (Bio-Rad, Tokyo, Japan) according to the manufacturer's instruction.

Peripheral blood mononuclear cells (PBMCs) were isolated from fresh EDTA venous blood samples by density gradient centrifugation (specific gravity, 1.077) (Histopaque 1077; Sigma-Aldrich).

### 4. Bovine Monocyte-Derived DCs

Bovine DCs were obtained as described elsewhere. Briefly, PBMCs were incubated with anti-human CD14-labelled super-paramagnetic particles (Miltenyi). CD14-expressing cells were positively enriched by passing the cells through a MidiMACS column (Miltenyi) according to the manufacturer instructions. CD14⁺ monocytes were cultured for 5 days in RPMI medium supplemented with 10% FBS, 0.5 mM 2-mercaptoethanol, 0.1 mM non essential amino acids, 50 μg/ml streptomycin and 50 IU/ml penicillin (all from Gibco-Invitrogen), recombinant bovine IL-4 and granulocyte-macrophage colony-stimulating factor (GM-CSF; both cytokines from Serotec).

### 5. Maturation of Bovine DCs

After 5 days of culture, immature bovine DCs were stimulated for 24 hours with 1 μg/ml anti-mouse, anti-human, or anti-bovine CD40 antibodies. Isotype control antibodies were used as negative controls. Lipopolysaccharide (LPS; 1 μg/ml) was used as a positive control of DC stimulation. Purified anti-bovine CD40 antibodies were heated for 2 hours at 56° C. to eliminate any LPS contamination. LPS-free anti-bovine CD40 antibodies were also used to stimulate bovine DCs. Supernatants were harvested at day 6 and IL-12 concentrations were measured by ELISA.

### 6. Quantification of IL-12 Levels

Bovine IL-12 levels were evaluated by ELISA. Briefly, 96-well plates were coated overnight at 4° C. with 4 μg/mL mouse anti-bovine IL-12 antibodies (clone CC301; Serotec) diluted in PBS. Plates were washed with PBS containing 0.05% Tween-20 (wash buffer) and blocked during 1 h with 1% BSA (Invitrogen) at room temperature. Plates were washed and 100 μL of sample was added for a 2-hour incubation. Plates were then washed and incubated for 1 hours with 100 μL of biotin-conjugated mouse anti-bovine IL-12 antibody (clone CC326; Serotec) diluted at 0.5 μg/mL. HRP-conjugated strepavidin (Serotec) was diluted in PBS (1:5,000) containing 1% BSA, and 100 μL of this solution was added to each well for 1 h, Trimethylbenzidine (TMB) substrate solution (100 μL, Sigma Chemical Co.) was added and the reaction was stopped after 30 min by the addition of 100 μL of 2M H2SO4. Absorbance was read at 450 nm.

### 7. Construction of Bovine CD40 Expression Vectors

An expression vector was designed to express the bovine CD40 receptor. Briefly, RNA was extracted from bovine thymic cells using TRIzol and was retrotranscribed into cDNA using a commercial kit (Roche Diagnostics, Mannheim, Germany). Two primers, F-CD40 and R-CD40, were designed based on the bovine CD40 cDNA sequence (GenBank accession number BC134765.1; National Center for Biotechnology Information (NCBI) Bethesda, Md., USA) to amplify a 843-bp fragment (F-CD40 5′-ATGGTTCGTTTGCCACTGCAG-3′ (SEQ ID NO:19) and R-CD40 5′-TCATTCTCGCTCCTGCACGG-3′ (SEQ ID NO:20)). The resulting polymerase chain reaction (PCR) product was cloned into an eukaryotic expression vector pcDNA3.1 (Invitrogen). The expression vector is hereafter referred to as pcDNA3.1/CD40. A two-step overlap extension PCR was used for generating another expression vector, the pcNA3.1/FLAGCD40, allowing the expression of a N-terminally FLAG-tagged CD40FLAG. This vector was constructed by inserting the nucleic acid sequence (GACTACAAGGACGACGATGACAAG (SEQ ID NO:21)) coding for a FLAG epitope (DYKDDDDK (SEQ ID NO:22)) between codons 20 and 21 of the CD40 open reading frame using the following primers:

F1-
(SEQ ID NO: 23)
TTTCTGACCGCCGTCCACTCAGACTACAAGGACGACGATGACAAGGAACC
AGCCACTGCTTGTGGAGAGA;
F2-
(SEQ ID NO: 24)
ATCGCCACCATGGTTCGTTTGCCACTGCAGTGTCTCTTCTGGGGCTTCTT
TCTGACCGCCGTCCACTCAGAC;
R-
(SEQ ID NO: 25)
ATAATCATTCTCGCTCCTGCACGGAGAT).

### 8. Mouse Immunization

Briefly, BALB/c mice were immunized by intramuscular injection of 100 μg of the pcDNA3.1/CD40 plasmid in 100 μl phosphate buffered saline (PBS) on days 0, 20 and 40. In order to boost the immune response, mice were injected on day 58 (viz. 4 days before the sacrifice) with 2×10⁷ NIH3T3 cells transfected with the pcDNA3.1/CD40 vector.

### 9. Cell Fusion

Hybridomas were produced by fusing spleen cells from the immunized BALB/c mice with P3X63Ag8.653 cells at a 3:1 ratio in a 50% solution of polyethylene glycol (Sigma) according to the standard procedure. The fused cells were selected in RPMI 1640 medium supplemented with hybridoma fusion and cloning supplement (Roche), 100 μM hypoxanthine-0.4 μM aminopterin-16 μM thymidine (HAT) supplement (Sigma), 2 mM L-glutamine, 1 mM sodium pyruvate, 0.1 mM non essential amino acids, 50 μM β-mercaptoethanol, 50 μg/ml streptomycin and 50 IU/ml penicillin (all from Gibco-Invitrogen). After 15 days, supernatants from wells with viable colonies were screened for the presence of anti-bovine CD40 antibodies by flow cytometry staining of bovine CD40-transfected COS-7 cells and immunoblotting of cell lysates prepared from bovine PBMCs (see below). The positive wells were cloned by flow cytometry sorting and monoclonal antibodies were purified on protein G affinity column (GE healthcare). The isotype of each monoclonal antibody was determined using a commercially available monoclonal antibody isotyping kit (Isotrip, Boehringer Mannheim Corp). For large scale anti-bovine CD40 production, hybridomas were amplified in a MiniPerm Bioreactor (Greiner Bio-One).

### 10. Western Blotting

Bovine PBMCs and COS-7 cells transfected with either the pcDNA3.1/CD40 vector or the empty pcDNA3.1 vector were lysed in a whole-cell lysis buffer (25 mM HEPES-KOH, 150 mM NaCl, 0.5% (v/v) Triton X-100, 10% (v/v) glycerol, 1 mM dithiothreitol (DTT), 1 mM Na₃VO₄, 25 mM β-glycerophosphate, 1 mM NaF and protease inhibitors (Complete, Roche)). Protein concentrations were measured with the Bio-Rad protein assay (Bradford reagent, BioRad). Equal amount of proteins were added to a loading buffer (10 mM Tris-HCl (pH 6.8), 1% SDS, 25% glycerol, 0.1 mM 2-ME, and 0.03% bromophenol blue), boiled, electrophoresed on a 12% polyacrylamide-SDS gel, and electrotransferred onto a nitrocellulose membrane (GE Healthcare). Nonspecific binding was blocked with 5% milk in PBS overnight at 4° C. Membranes were then incubated with polyclonal sera from immunized mice (dilution 1:5,000) or with hybridoma supernatants (dilution 1:500) for 120 min at room temperature, rinsed with 0.1% Tween 20/PBS and further incubated with horseradish peroxidase (HRP)-conjugated rabbit anti-mouse IgG (Dako, Germany) for 60 min at room temperature. Result was visualized using an enhanced chemiluminescence system (GE Healthcare) followed by autoradiography with Fuji x-ray films.

### 11. Flow Cytometry Staining for Cell Surface Expression of CD40

To check whether hybridomas were able to produce monoclonal antibodies directed to bovine CD40, hybridoma supernatants were collected and used to detect CD40 expression by COS-7 cells transfected with the pcDNA3/CD40 vector. 10⁶ CD40-transfected COS-7 cells were incubated for 15 min (at 4° C.) with 100 μl of hybridoma supernatants. COS-7 cells transfected with the empty pcDNA3.1 vector were used as controls. Cells were washed twice in FACS buffer (PBS, 1% bovine serum albumin (BSA), 0.1% sodium azide) and were incubated with 0.5 μl of FITC-conjugated goat anti-mouse IgG (Serotec). Cells were then washed and flow cytometry analysis was performed with a FACScanto (BD Biosciences).

### 12. Cow Immunization

Eighteen healthy Holstein heifers were randomly allocated into three groups of six animals. The first group was injected subcutaneously in the right prescapular region with 10⁹ CFUs of heat-killed S. aureus (HKSA) and 5 mg of the E1 monoclonal anti-bovine CD40 antibody. The second group was injected with HKSA alone and the third group was left untreated. HKSA was prepared as described above.

### 13. Restimulation of Prescapular Lymph Nodes Cells

Five days after immunization, prescapular lymph nodes (PLN) were biopsied. PLN cells were isolated and cultured in RPMI medium supplemented with 10% heat-inactivated bovine serum and additives (2 mM L-glutamine, 1 mM sodium pyruvate, 0.1 mM non essential amino acids, 50 μM β-mercaptoethanol, 50 μg/ml streptomycin and 50 IU/ml penicillin). PLN cells were left untreated or restimulated with either 1×10⁵ HKSA or 50 μg/ml ovalbumin (OVA; used as an irrelevant antigen). The proliferation of PLN cells was measured as ³H-thymidine incorporation during the last 16 h of a 3-day culture. Culture supernatants were assayed for bovine IFN-γ and IL-4 by ELISA.

### 14. Intracellular Bovine IFN-γ Staining

PLNs cells were stimulated in vitro with 1×10⁵ HKSA/ml for 20 hours and incubated with Brefeldin (GolgiPlug, BD Biosciences) A for the last 4 hours. Cells were stained for either CD4 or CD8. Cells were then fixed and permeabilized with BD Cytofix/Cytoperm™ Fixation/Permeabilization Kit (BD Biosciences), and stained intracellularly with an Alexa-647 conjugated anti-bovine-IFN-γ Ab (Serotec). Cells were then washed twice in a FACS buffer and flow cytometry analyses were performed with a FACScanto (BD Biosciences).

### 15. Statistical Analysis

Data are presented as means±standard deviations (SDs). The differences between mean values were estimated using an analysis of variance test followed by a Fisher's protected least standard deviation test. A value of P<0.05 was considered significant. All the experiments were repeated at least three times.

### Results

### 1: Agonistic Anti-Mouse and Anti-Human CD40 Antibodies Fail to Induce IL-12 Secretion by Bovine DCs.

Engagement of CD40 on the surface of DCs stimulates them to produce pro-inflammatory cytokines such as IL-12, and therefore promotes DC-mediated T cell activation [1]. To determine whether agonistic anti-mouse or anti-human CD40 antibodies could be used to stimulate the bovine CD40 receptor, we generated monocyte-derived bovine DCs and stimulated them with commercial antibodies directed to mouse and human CD40. LPS was used as a positive control for induction of IL-12 secretion. As shown in FIG. 7, none of the antibodies tested was able to induce IL-12 production by bovine DCs, indicating that generation of an agonistic anti-bovine CD40 antibody was required for efficient stimulation of the bovine CD40 receptor.

### 2: Generation of Anti-Bovine CD40 Monoclonal Antibodies.

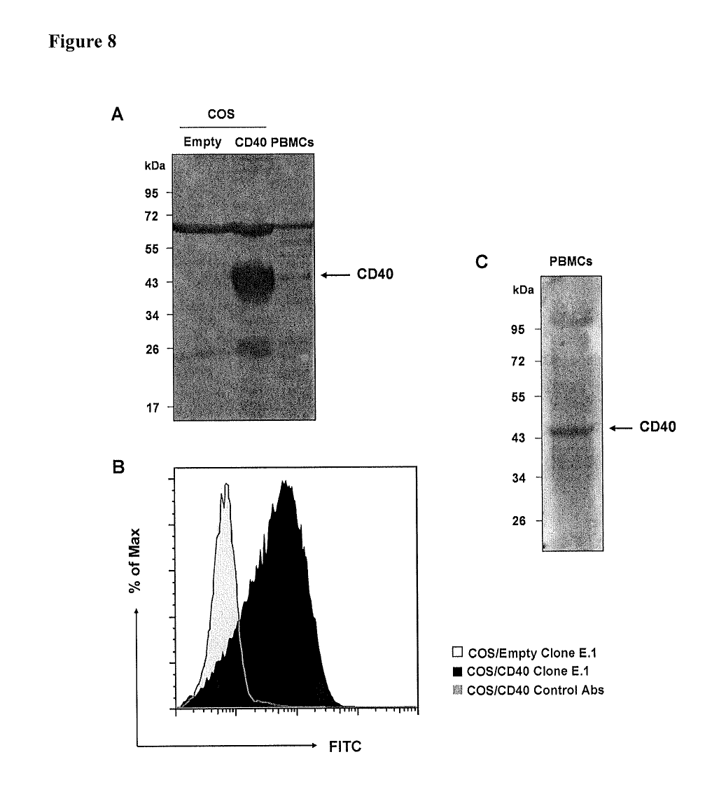

To generate specific monoclonal antibodies directed to bovine CD40, a plasmid DNA expression vector for bovine CD40 was constructed (this vector is hereafter referred to as pcDNA3.1/CD40). Given the absence of commercial bovine anti-CD40 antibodies for detection, a FLAGFLAG epitope was alternatively added to the N terminus of the protein (the modified vector was called pcDNA3.1/FLAGCD40). Using anti-FLAG antibodies and flow cytometry analyses, we observed that transfection of NIH3T3 cells with the pcDNA3.1/FLAGCD40 vector allowed efficient membrane expression of the bovine CD40 protein in these cells (data not shown). Mice were then immunized on days 0, 20, and 40 by intramuscular injection of the pcDNA3.1/CD40 plasmid, encoding the untagged CD40 protein. In order to boost the immune response, mice were injected on day 58 with 2×10⁷ NIH3T3 cells transfected with the pcDNA3.1/CD40 vector. To test whether the serum of immunized mice contained antibodies directed against bovine CD40, lysate from bovine PBMCs, which are known to spontaneously express CD40, and CD40-transfected COS-7 cells were subjected to western blot analysis using mice sera as primary antibodies. As shown in FIG. 8a, serum polyclonal antibodies recognized a protein weighing 45 kDa, which is the expected size of bovine CD40. Using splenocytes from these mice, a large number of mouse-bovine hybridomas producing monoclonal antibodies were produced by standard procedures. In order to find out whether these monoclonal antibodies were able to recognize the bovine CD40, a surface staining of bovine CD40-transfected COS-7 cells was performed using flow cytometry. Among the monoclonal antibodies tested, eight were found to detect the bovine CD40 (i.e. the AA5, E1, M.1, OO.1, H1, 2W.1, K.1 and 2N clones). A representative example (i.e. the results obtained with the E1 antibody) is provided in FIG. 8b. To confirm the recognition of the endogenously expressed protein, lysates from bovine PBMCs were subjected to western blot analysis using the purified monoclonal antibodies as primary antibodies. The eight antibodies allowed the detection of the CD40 protein. The results obtained with the E1 antibody are shown in FIG. 8c as an example. Immunoglobulin isotype analysis revealed that AA5 and E1 anti-CD40 antibodies were IgG_{2b}, whereas M.1, OO.1, H1, 2W.1, K.1 and 2N antibodies were IgG₁.

### 3: The E1 Monoclonal Antibody is Able to Strongly Induce IL-12 Secretion by Bovine DCs.

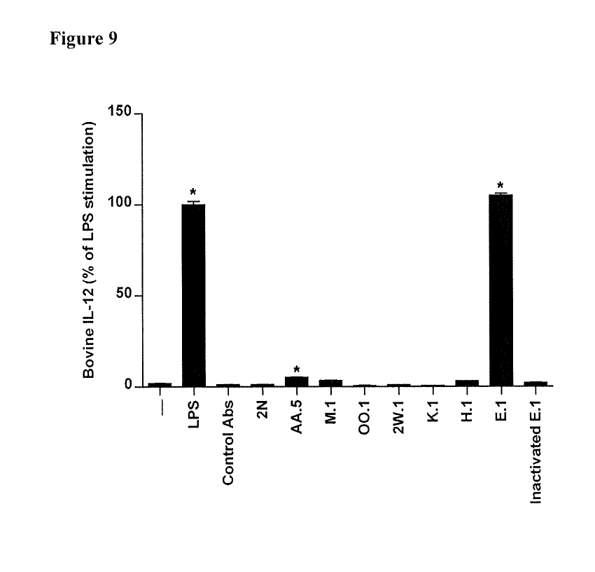

To determine whether monoclonal anti-bovine CD40 antibodies we had generated displayed agonistic properties, we used them to stimulate bovine DCs. LPS was used as a positive control of DC activation. As shown in FIG. 9, the E1 antibody was capable of strongly inducing IL-12 production by bovine DCs. Indeed, when this antibody was used, the levels of IL-12 measured in the supernatant of DCs were comparable to those measured in the supernatant of LPS-stimulated DCs. Of note, the AA5 antibody slightly, but significantly, promoted IL-12 production by DCs (FIG. 9). To rule out any LPS contamination of the E1 monoclonal antibody, the E1 samples were heated for 2 hours at 56° C. This procedure inactivates the antibodies but not the LPS, which is not thermosensitive. FIG. 9 shows that E1 samples that were heat-inactivated were no longer able to induce IL-12 secretion by DCs, demonstrating that the E1 antibody was not LPS-contaminated.

4: Immunization with E1 Monoclonal Antibodies Associated with HKSA Primes Antigen-Specific IFN-γ-Producing CD8⁺ T Cells In Vivo.

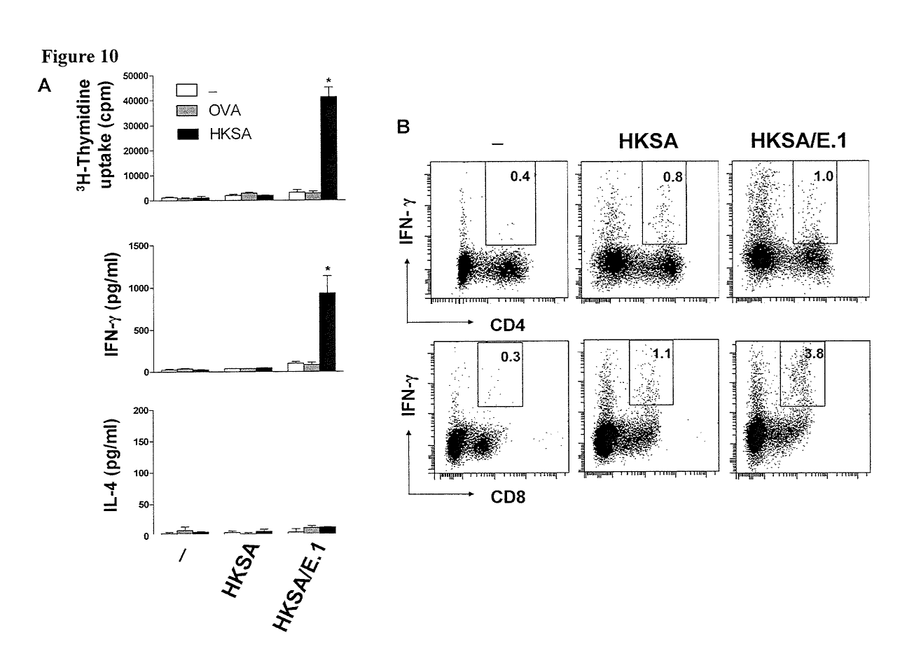

As demonstrated above, combining agonistic anti-CD40 antibodies with HKSA is an efficient approach for inducing HKSA-specific CTL responses in mice. We therefore tried to determine whether it was possible to induce CTL responses against HKSA in healthy cows. For that, HKSA alone or associated with the E1 monoclonal antibody was injected subcutaneously in the right prescapular area. Five days later, prescapular lymph nodes (PLN) cells were collected and restimulated in vitro with HKSA or OVA (used as an irrelevant antigen). PLN cells from cows that were injected with E1 antibodies and HKSA exhibited a significant proliferative response in the presence of HKSA, whereas no or little proliferative response was observed in PLN cells from untreated cows or cows that received HKSA alone (FIG. 10a). To better characterize the immune response induced by co-administration of E1 antibodies and HKSA, the concentrations of bovine IFN-γ and IL-4 in the supernatant of HKSA-restimulated PLN cells were assessed by ELISA. Low cytokine levels were detected in the supernatant of PLN cells from untreated and HKSA-treated cows (FIG. 10a). In contrast, immunization with HKSA and E1 antibodies was associated with very high levels of IFN-γ production (FIG. 10a). As shown in FIG. 10a, restimulation of PLN cells from cows that were immunized with the combination of HKSA and E1 antibodies did not respond to OVA stimulation, demonstrating that the immune response was antigen-specific in these animals.

To clearly identify the cellular source of IFN-γ, we next performed intracellular staining experiments. As described above, cows were immunized and PLN cells were restimulated with HKSA. CD4⁺ and CD8⁺ cells were then stained intracellularly for bovine IFN-γ. Although some IFN-γ-secreting CD4⁺ T cells were found in the PLNs from cows immunized with HKSA associated with the E1 antibody, the major source of IFN-γ in these cows was the CD8⁺ T cell population (FIG. 10b). CD4⁺ and CD8⁺ T cells from untreated animals and animals immunized with HKSA alone produced only small amounts of IFN-γ (FIG. 10b). These data show that immunization of cows with HKSA together with the monoclonal anti-bovine CD40 antibody promotes the development, in the local draining lymph nodes, of IFN-γ-secreting CD8⁺ T cells specifically directed against HKSA.

### Conclusion

Vaccination is generally considered to be the most effective strategy to prevent infectious diseases in cattle, and is an obvious alternative to the use of antibiotics. However, commonly used adjuvants (aluminium hydroxide or oils) predominantly promote humoral responses and there is currently no available adjuvant able to induce strong CTL responses to intracellular bovine pathogens. Among the new molecular adjuvants tested in the mouse, the combination of agonistic anti-CD40 antibodies and Toll-like receptor (TLR) agonists seems the most potent approach to induce CTL responses and to prevent intracellular infections. We therefore hypothesized that such an approach could also help in the control of intracellular infections in cattle. To verify our hypothesis, we have generated an agonistic monoclonal anti-bovine CD40 antibody (i.e. the E1 antibody), and have demonstrated that combining this antibody with HKSA, an exogenous antigen capable of stimulating TLR2 and TLR4, results in the expansion of antigen-specific IFN-γ secreting CD8⁺ T cells in the draining lymph nodes of immunized cows.

In this study, eight different hybridomas were found to produce monoclonal antibodies able to bind the bovine CD40 expressed on COS-7 cells. Among these antibodies, the E1 antibody induced strong production of IL-12 by bovine DCs, which is the signature of agonistic properties. Indeed, ligation and activation of CD40 receptor has been reported to induce the maturation of DCs, as reflected by increased expression of costimulatory and MHC molecules, and by enhanced production of proinflammatory cytokines production as IL-12. Interestingly, it has been shown that CD40-activated DCs do not require Th1 cells as intermediary cells to induce CTL responses. [5] In this case, Th1 cells are therefore bypassed. Moreover, IL-12 produced by CD40-activated DCs can directly act on CD8⁺ T cells to promote their proliferation, survival and differentiation into effector and memory CTLs. In the present study, injection of the E1 antibody to experimental cows induced selective development of antigen-specific IFN-γ-secreting CD8⁺ T cells in the draining lymph nodes. We therefore conclude that the E1 antibody instructed local DCs to preferentially induce CTL responses.

It has already been shown in other models that injection of an exogenous antigen together with agonistic anti-CD40 antibodies may induce effector OVA-specific CD8⁺ T cells. In accordance with these previous studies, we have shown that the E1 monoclonal antibody is able to boost the development of strong CTL responses directed against HKSA in cows, whereas HKSA alone only induced poor immunity. It has been demonstrated that induction of efficient CTL responses by agonistic anti-CD40 antibodies requires concomitant stimulation of TLRs. Interestingly, TLR2 and TLR4 are bound and activated by HKSA, suggesting that two concomitant mechanisms favoured the development of CTL responses in our model. First, the activation of CD40 in DCs by the E1 antibody. Second, the stimulation of TLR2 and TLR4 by the HKSA antigen itself.

S. aureus is a major pathogen involved in chronic bovine mastitis. S. aureus remains difficult to control due to its ability to invade and survive within host cells, including mammary epithelial cells. We have shown in a mouse model of S. aureus-induced mastitis that combining CD40 activation with HKSA immunization is an efficient approach for vaccinating against staphylococcal mastitis. Now that we have generated an agonistic monoclonal anti-bovine CD40 antibody (i.e. the E1 antibody), it will be interesting to test this approach in cow. Moreover, the E1 antibody could also be tested for its ability to induce CTL responses against other intracellular bovine pathogens, including parasites, viruses or bacteria. For example, Streptococcus uberis represents another important emerging bovine mastitis pathogen and is also reported in chronic mastitis. As observed for S. aureus, S. uberis can persist inside bovine mammary epithelial cells and would therefore require efficient CTL responses to be cleared.

The E1 antibody belongs to the IgG2b subclass, which does not fix complement nor binds to Fc receptors effectively, suggesting that most of the biological effects of the E1 antibody were dependent on its antigen-specific variable region rather than on its constant region. However, as the E1 antibody has been generated in the mouse, it could become immunogenic in the bovine species and generate bovine anti-mouse antibody responses. This problem could easily be circumvented by generating a <<bovinized>> version of the E1 antibody.

In conclusion, we have generated a monoclonal anti-bovine CD40 antibody which exerts agonistic properties. We furthermore showed that combining the E1 antibody with HKSA led to the development of a strong and antigen-specific CTL response in vivo. We conclude that the E1 antibody can be used as an adjuvant to induce CTL responses in cattle and to prevent staphylococcal mastitis, but also other infectious diseases involving intracellular pathogens.

### Example III

### Administration of Agonistic Anti-CD40 Antibody in the Vaccination Murine Mouse Model: Increased Humoral Response to HSKA as Mentioned by Antibody Titers of Anti-HSKA Antibodies

### Material and Methods

### ELISA Measurements of the Ag-Specific Ig Titers

Antibodies against HKSA were evaluated by ELISA in microtiter plates coated with 10⁷ CFU of HKSA per well. Diluted sera from immunized mice were incubated on Elisa plates and bound IgG2a and IgG2b were detected using horseradish peroxidase (HRP)-conjugated mouse IgG2a and IgG2b specific antibodies (Southern Biotechnology) followed by incubation with tetramethyl benzidine and measurement by spectrophotometry. Antibody titers were calculated by plotting the serum dilution that gave half-maximal signal. When no signal was detected, we assigned a titer of 2.

### Results

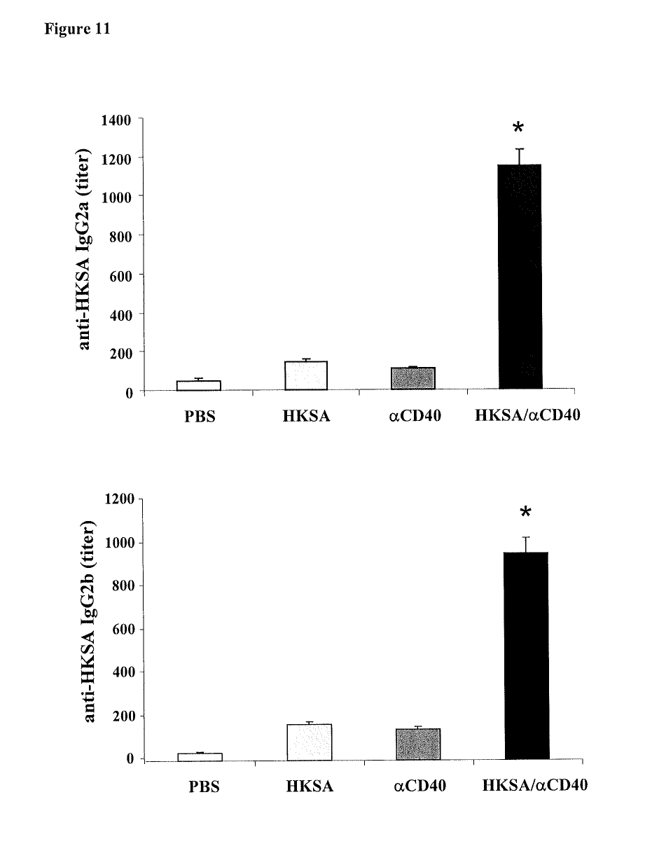

The specific humoral response to HKSA was also measured. As IgG2a and IgG2b are the most representative immunoglobulins of a Th1 type immune response, we mainly focused on these 2 isotypes. For these 2 tested isotypes, immunization of mice with HKSA/αCD40 led to significantly higher titers of anti-HKSA antibodies compared with mice treated with either PBS, HKSA alone, or αCD40 alone (FIG. 11). These results show that immunization with HKSA alone is not able to trigger an efficient humoral response by itself and that immunization of mice with HKSA combined with agonistic anti-CD40 antibodies induces the development of a strong Th1 immune response.

### Example 4

### Development of Vectors Encoding the Bovine CD40L, Production of Recombinant Bovine CD40L in Prokaryotic and Eukaryotic Cells and Use of these Proteins and Vectors in Vaccine Strategy Aimed at Preventing or Treating Bacterial Mastitis in Cows

### Material and Methods:

### 1. Cloning of the Different Forms of Bovine CD40L

RNA was extracted from bovine blood mononuclear cells using Trizol (Invitrogen) and retrotranscribed into cDNA using a commercial kit (Roche Diagnostics, Mannheim, Germany). Primers were designed based on the bovine CD40L cDNA sequence (GenBank accession number Z48469; National Center for Biotechnology Information (NCBI) Bethesda, Md., USA) to amplify the different fragments by polymerase chain reaction (PCR). The sequences of the primers used in this study are displayed in Table 1.

TABLE 1
list of primers used to construct the different forms of bovine CD40L

| | | |
|---|---|---|
| 1 | Bov_CD40L_EC_F | CACCCTTCACAGACGATTGGACAAG (SEQ ID NO: |
| | | 28) |
| 2 | Bov_CD40L_solEC_F | CACCATGCACAAGGGTGATCAGGA (SEQ ID NO: |
| | | 29) |
| 3 | Isol_CD40L_EC_F | CACCAGAATGAAGCAGATCGAGGACAAGATCGAG |
| | | GAGATCCTGAGCAAGATCTACCACATCGAGAACG |
| | | AGATCGCCAGAATCAAGAAGCTGATCGGCGAGAG |
| | | AACCAGCAGCCACAGACGATTGGACAAGATA |
| | | (SEQ ID NO: 30) |
| 4 | Isol_CD40L_solEC_F | CACCAGAATGAAGCAGATCGAGGACAAGATCGAG |
| | | GAGATCCTGAGCAAGATCTACCACATCGAGAACG |
| | | AGATCGCCAGAATCAAGAAGCTGATCGGCGAGAG |
| | | AACCAGCAGCATGCACAAGGGTGATCAGGA (SEQ |
| | | ID NO: 31) |
| 5 | Bov_CD40L_R | TCAGAGTTTGAGTAAGCCAAAT (SEQ ID NO: 32) |
| 6 | GST_Hind3_F | AAGCTTTCCCCTATACTAGGTTATTGG (SEQ ID NO: |
| | | 33) |
| 7 | Bov_CD40L_EcoRI_R | GAATTCTCAGAGTTTGAGTAAGCCAAAT (SEQ ID |
| | | NO: 34) |
| 8 | Bov_CD40L_EC_Hind3_F | AAGCTTCACAGACGATTGGACAAGATAG (SEQ ID |
| | | NO: 35) |
| 9 | CD40L_solEC_Hind3_F | AAGCTT ATGCACAAGGGTGATCAGGAG (SEQ ID |
| | | NO: 36) |
| 10 | Isol_CD40L_Hind3_F | AAGCTTAGAATGAAGCAGATCGAGGAC (SEQ ID |
| | | NO: 37) |
| 11 | NheI_prepro_FLAG_Hind3_isol_F | GCTAGCTCACCATGCATCCCCTGCTTATCCTTGCCT |
| | | TTGTGGGAGCTGCTGTGGCTGACTACAAAGACGAT |
| | | GACGACAAGCTTAGAATGAAGCAGATCGAGGAC |
| | | (SEQ ID NO: 38) |
| 12 | GST_R | GAATTCTCAACGCGGAACCAGATCCGATTT (SEQ |
| | | ID NO: 39) |

In order to produce the different forms of bovine CD40L in bacteria, four different PCR fragments corresponding either to the total extracellular form or to the natural soluble form of bovine CD40L or to these forms preceded by an isoleucine motif encouraging trimerization were generated by using the couple of primers 1 and 5, 2 and 5, 3 and 5, 4 and 5, respectively. These fragments were cloned into the prokaryotic vector pDEST15 (Invitrogen) which allows the fusion of glutathione S transferase at the N terminal part of each CD40L forms.

For the production of proteins in mammalian cells, two different expression vectors were used: pcDNA3.1 (Invitrogen) and pFLAG-CMV3 (Sigma). Similar PCR fragments as described above were generated by using either the couple of primers 6 and 7 with each respective pDEST15 vector as template DNA to get GST fusion proteins or the couple of primers 8 and 7, 9 and 7, or 10 and 7 with each respective pDEST15 vector as template. The different fragments were cloned into the pFLAG vector. For pcDNA3.1 constructions, the PCR fragments coding for the different forms of CD40L were generated by first using the couple of primers 11 and 7 with pDEST15-isol-ECform and pDEST15-isol-solECform as template and secondly the couple of primers 8 and 7 or 9 and 7 with the same templates. These fragments have been cloned into pcDNA3.1 to get the 2 extracellular forms with or without the trimerization motif preceded by a FLAG and the signal peptide for preprotrypsinogen. Moreover, some PCR fragments were generated by using the couple of primers 6 and 7 with each forms of pDEST15 as template. These fragments were cloned into the pcDNA3.1-prepro-FLAG-ECform by replacing the sequence coding for the ECform region by the PCR fragments leading to constructions coding for the 2 extracellular forms of CD40L with or without the trimerization motif preceded by the GST sequence, a FLAG and the signal peptide for preprotrypsinogen. For controls, an empty pDEST15 vector was used for bacterial protein production and a PCR fragments generated by using the couple of primers 12 and 7 was cloned into pFLAG and pcDNA3.1 in order to produce the protein GST alone. The detailed list of all the constructions that have been generated is displayed in Table 2.

TABLE 2
List of the different constructions generated for recombinant CD40L production

| | | | Sequence |
|---|---|---|---|
| Production | | | (amino acids of |
| type | Vectors | CD40L form type | SEQ ID NO: 27) |
| Prokaryotic | pDEST15 | CD40L_ECform | 46-261 |
| protein | (contains a | CD40L_solECform | 113-261 |
| production | GST at N- | Isol_CD40L_ECform | 47-261 |
| | terminal) | Isol_CD40L_solECform | 113-261 |
| | | — | — |
| Eukaryotic | pFLAG | CD40L_ECform | 47-261 |
| protein | | CD40L_solECform | 113-261 |
| production | | Isol_CD40L_ECform | 47-261 |
| | | Isol_CD40L_solECform | 113-261 |
| | | GST_CD40L_ECform | 46-261 |
| | | GST_CD40L_solECform | 113-261 |
| | | GST_Isol_CD40L_ECform | 47-261 |
| | | GST_Isol_CD40L_solECform | 113-261 |
| | | GST | — |
| | pcDNA3.1 | Prepro-FLAG-CD40L_ECform | 46-261 |
| | | Prepro-FLAG-CD40L_solECform | 113-261 |
| | | Prepro-FLAG-Isol_CD40L_ECform | 47-261 |
| | | Prepro-FLAG-Isol_CD40L_solECform | 113-261 |
| | | Prepro-FLAG-GST_CD40L_ECform | 46-261 |
| | | Prepro-FLAG-GST_CD40L_solECform | 113-261 |
| | | Prepro-FLAG-GST_Isol_CD40L_ECform | 47-261 |
| | | Prepro-FLAG-GST_Isol_CD40L_solECform | 113-261 |
| | | Prepro-FLAG-GST | — |

### Protein Production in Eukaryotic Cells:

### Transfection of Cos7 Cells

COS-7 cells were obtained from the American Type Culture Collection (Rockville, USA). The cells were maintained in RPMI 1640, supplemented with 2 mM L-glutamine, 1% MEM non essential amino acids and 10% foetal bovine serum. For transient transfection, cells were seeded in 175 cm² tissue culture dishes 24 h prior to transfection in order to reach 95% of confluence the next day. Plasmids (pcDNA3.1 or pFLAG vectors) were transfected using TransFectin reagents (Bio-Rad, Tokyo, Japan) according to the manufacturer's instruction. The volume of culture medium covering the cells was only 10 ml in order to concentrate the best possible the proteins secreted in the medium. The medium was recovered 32 h after the time of transfection and stocked at −80° C. before being tested for the presence of proteins by ELISA.

### Anti-GST Elisa

The GST-fusion proteins were detected with an anti-GST ELISA according to the manufacturer's instructions (Pierce Biotechnology, Rockford, USA).

### Anti-FLAG ELISA

The proteins coupled to a FLAG were detected with an anti-FLAG ELISA according to the manufacturer's instructions (Sigma, Saint-Louis, USA). Two different detection antibodies were used in parallel as a double control: an anti-CD40L antibody (Santa Cruz, Heidelberg, Germany) and an anti-GST antibody (Pierce Biotechnology, Rockford, USA).

### Functionality Test

The protein produced in the supernatant have to be tested for their functionality, for their agonist properties. We have developed a test based on the fact that specific activation of the CD40 receptor at the surface of bovine endothelial aortic cells leads to the production of the chemokine MCP-1. Thus each of the supernatant produced by Cos7 cells has been evaluated in this test. Briefly, bovine endothelial aortic cells were cultured in low glucose DMEM medium supplemented with 2 mM L-glutamine, 1% MEM non essential amino acids, 1 ng/ml of fibroblast growth factor and 10% foetal bovine serum. The cells were seeded in 48 wells culture dishes and were stimulated with IFN-γ (10 ng/ml) during 20 h in order to upregulate CD40 receptor expression. Then medium was recovered from the cells and replaced by the supernatant of Cos7 cells containing our proteins. Sixteen hours later, culture medium was recovered to measure MCP-1 production by ELISA according to the manufacturer's instructions (Kingsfisher Biotech, St Paul, USA). The endothelial cells are very sensitive to LPS contamination. Thus, to determine whether the measured effect on MCP-1 production is due to LPS contamination, a part of the medium was boiled before being putted on the cells. Boiling denatured the protein and reduced strongly MCP-1 production if no LPS was present. Moreover, the boiling provided a supplemental control that inactivated the protein agonistic capacity.

### Protein Production in Bacteria:

### Transformation and Culture of Bacterial Cells

An aliquot of BL21-A1 one shot competent cells (Invitrogen) was transformed with plasmids pDEST15 by heat shocking according to the manufacturer's instructions. They were spread on LB plates containing 100 μg/ml ampicillin and incubated overnight at 37° C. Three transformants were picked and cultured in LB medium containing 100 μg/ml ampicillin at 37° C. under shaking until reaching an OD of 0.8. This culture was used to inoculate 300 ml of fresh LB culture medium containing 100 μg/ml ampicillin. This culture was incubated until an OD of 1. Then L-arabinose was added to the medium to induce protein production and the culture was incubated under shaking during 5 hours at 20° C.

### Protein Extraction

After an incubation of 5 h, the culture medium was centrifuged at 5000 g during 10 minutes and the bacterial pellet was resuspended and incubated at 4° C. during 40 minutes in a lysis buffer containing NaCl 0.5M, NaH₂PO₄ 50 mM pH 8.0, 1.8 mg/ml lyzozym and 20 μg/ml DNAse. After incubation, 0.5% of Triton X-100, complete protease inhibitor (Roche, Mannheim, Germany), and β-glycerphosphate 30 mM were added. The solution was then sonicated 6 times for 0.5 minutes each. The solution was then centrifuged at 20000 g for 20 minutes; and the supernatant containing the proteins was recovered and passed through a 0.2 μm filter.

### Western-Blot

The presence of proteins in extracts was studied by western-blotting. Equal amount of protein extracts were added to a loading buffer (10 mM Tris-HCl pH 6.8, 1% SDS, 25% glycerol, 0.1 mM β-mercaptoethanol, and 0.03% bromophenol blue), boiled and electrophoresed on a 14% polyacrylamide-SDS gel. After electrotransfer onto a nitrocellulose membrane (Amersham Pharmacia Biotech, Roosendaal, Netherlands), nonspecific binding was blocked in 20 mM Tris, pH 7.5, 500 mM NaCl, 0.2% Tween 20 (TBS-Tween), and 5% dry milk during 1 hour. Membranes were then incubated with polyclonal anti-GST (dilution 1:5,000) overnight at 4° C., rinsed with TBS-Tween and further incubated with horseradish peroxidase (HRP)-conjugated rabbit anti-mouse IgG (Dako, Germany) for 60 min at room temperature. The result was visualized using an enhanced chemiluminescence system (ECL kit; Amersham Pharmacia Biotech, Roosendaal, Netherlands) followed by autoradiography with Fuji x-ray films.

### Protein Purification

Proteins present in the lysis buffer supernatant were purified using glutathione agarose resin columns (Pierce Biotechnology, Rockford, USA). Briefly, columns were equilibrated with equilibration buffer (150 mM Nacl, 125 mM Tris pH8). The supernatant containing the proteins was loaded on the columns and binding was achieved by incubation with the resin during 1 hour under shaking at room temperature. The supernatant containing the unbound proteins was discarded and column was washed 4 times with wash buffer (150 mM Nacl, 125 mM Tris pH8, Triton x-100 0.55%). Then a wash was performed with a similar buffer containing 0.45% Triton x-100 and a last wash with the same buffer without Triton x-100. Elution was performed with a buffer containing 300 mM Nacl, 125 mM Tris pH8.75 and glutathione 3 mg/ml.

Eluates were detoxified using EndoTrap Blue columns (Hyglos, bernried, Germany) according to the manufacturer's instructions.

### Protein Dosage

The quantity of purified proteins was estimated in an anti-GST Elisa according to the manufacturer's instructions (Pierce Biotechnology, Rockford, USA).

### Functionality of the Proteins

Eluates containing the proteins were tested in the functional test as described before with little modifications. Briefly, bovine aortic endothelial cells were seeded in 48 wells culture dishes and were stimulated with IFN-γ (10 ng/ml) for 20 h. Then cells were stimulated with a few quantities of the boiled or non-boiled detoxified eluates containing our proteins for 16 h. The culture medium was then recovered and tested for MCP-1 production by ELISA according to the manufacturer's instructions (Kingsfisher Biotech, St Paul, USA).

### Verification of Ligation Capacity of the Ligand

In order to determine whether the ligand ligates correctly to the receptor, we transfected Cos7 cells with a plasmid coding for bovine CD40 receptor or plasmic not coding for bovine CD40 and evaluate by flow cytometry the ligation of CD40L. Briefly, transfected Cos7 cells were incubated with GST proteins for control or CD40L proteins for 90 minutes. They were then washed and incubated with anti-GST antibodies for 75 minutes. They were washed and incubated with goat PE-anti-rabbit IgG (Imgenex) for 1 hour. The samples were then washed and analysed by flow cytometry (FACS Canto II, Becton Dickinson).

### Results

### Prokaryotic Production

### Constructions for Production of Recombinant CD40L Protein in Bacteria

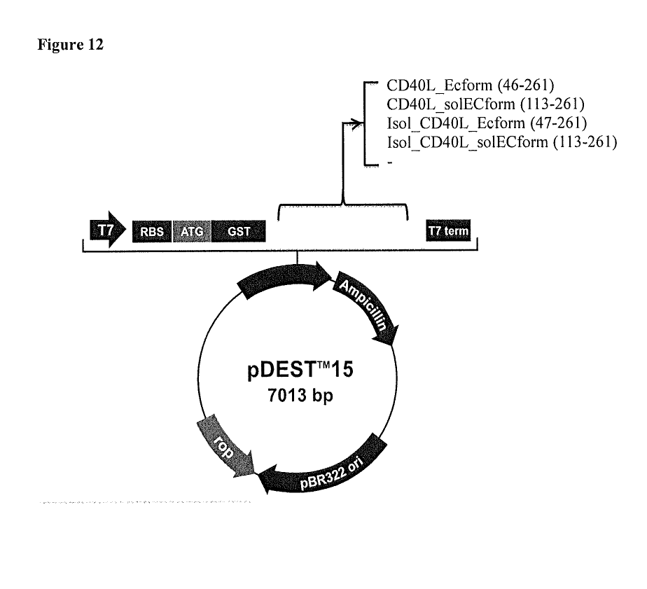

For production of CD40L in bacteria, the PCR fragments coding for the different forms of CD40L were cloned into pDEST15 vectors (FIG. 12).

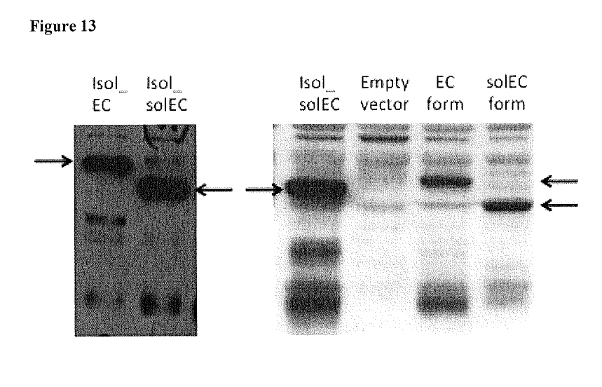

BL21-A1 one shot competent cells were transformed with the different plasmids and protein production was induced by stimulation with arabinose as described previously. Then proteins were extracted in native condition as described in above; and the presence of proteins was assessed by western-blot using anti-GST antibodies. We can see in FIG. 13 that all the constructions are functional and that all the CD40L forms have been produced by BL21-A1 cells.

### Purification of the Proteins Produced in Bacteria

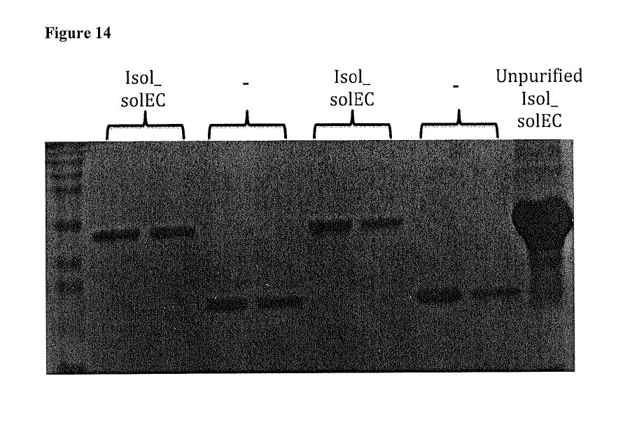

CD40L proteins were purified on anti-GST affinity columns. The purified proteins were then loaded on an electrophoresis gel which was stained by coomassie blue. FIG. 14 shows the coomassie blue-stained gel presenting the purified soluble extracellular form of CD40L preceded by the trimerization motif and fusionned to GST (i.e. Isol_solEC) and the purified GST protein alone (i.e. -) in comparison with unpurified Isol_solEC form. The eluates containing purified proteins were detoxified using EndoTrap Blue columns.

### Protein Dosage

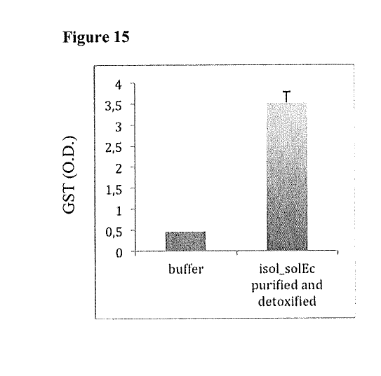

The eluates containing purified and detoxified proteins were tested in a GST ELISA to estimate the quantity of protein production. We can see in the FIG. 15 that we can detect easily our proteins in the eluates by ELISA.

### Functional Challenge of the Purified Proteins

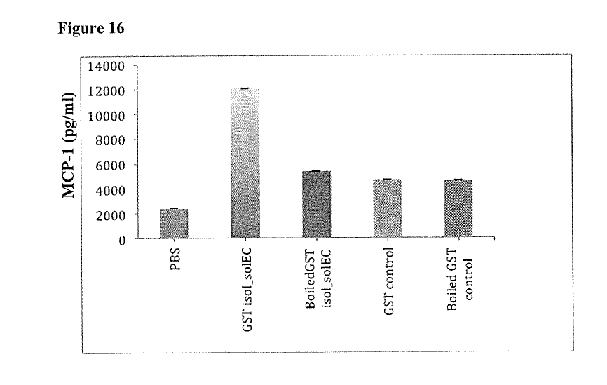

The purified and detoxified proteins were then challenged in the functionality test that is based on CD40 receptor stimulation at the surface of bovine aortic endothelial cells and measurement of the subsequent MCP-1 production by ELISA. In order to evaluate the specificity of the stimulation, the eluates containing the GST fusion-CD40L were systematically compared to eluates that just contained GST proteins. Moreover as our test is very sensitive to LPS, we compared the eluates of interest with boiled eluates in order to verify if the observed effect in MCP-1 production was not due to LPS stimulation of the cells. FIG. 16 shows the production of MCP-1 by endothelial cells stimulated with either the eluate containing the soluble extracellular form of CD40L preceded by a trimerization motif in comparison with the same eluate which has been boiled, or for control, eluate containing GST proteins in comparison with the same eluate which has been boiled. We can see that stimulation of cells with the eluate containing our proteins CD40L induced a significant production of MCP-1 when compared with cells stimulated with the control GST proteins. This effect is abolished when the eluate is boiled before stimulation for CD40L proteins, while boiling has no effect for GST control proteins confirming that our effect is specific of our CD40L proteins and that this effect is not due to LPS stimulation.

In order to improve protein yields and the retrieve of LPS, we used Triton x-114 during the last washes of the purification process. Essentially, we replaced Triton x-100 in the penultimate wash by Triton x-114 and also added it in the last wash and in the elution buffer. As Triton x-114 has the characteristic to precipitate above 22° C., we made the last washes and elution at 4° C. and then precipitated the Triton x-114 by centrifugation at 37° C. to remove it from the eluate. We then performed an agonistic test and performed a MCP-1 ELISA (FIG. 23). The results show that the difference in MCP-1 production between GST and GST fusion CD40L is increased by using Triton x-114 when compared with FIG. 16, due to a better detoxification of the eluate.

### Ligation Capacity of CD40L Proteins to CD40 Receptor

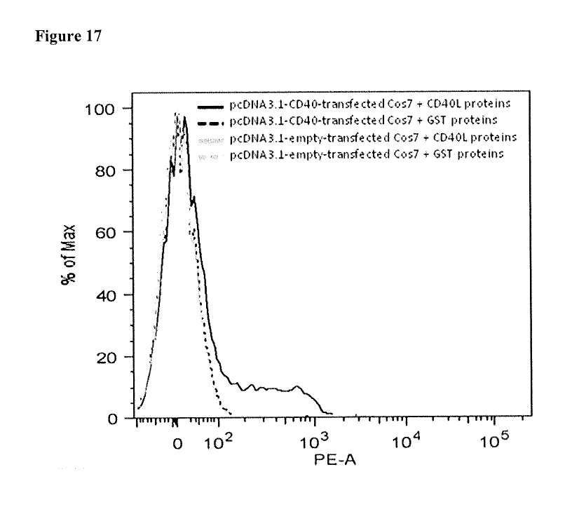

As described before, Cos7 cells were transfected with plasmids coding for bovine CD40 receptor (black line) or not encoding for CD40 receptor (grey line) and incubated with GST or GST fusion CD40L proteins. Then the cells were washed and incubated with anti-GST antibodies followed by PE-anti-rabbit IgG. Cells were then analyzed by flow cytometry. FIG. 17 shows the intensity of fluorescence of cells incubated with GST proteins (dotted line) or GST-fusion CD40L proteins (continuous line). These results confirmed the specific ligation of CD40L proteins to bovine CD40 receptor.

### Eukaryotic Production

### Constructions for Production of Recombinant CD40L Protein in Mammalian Cells

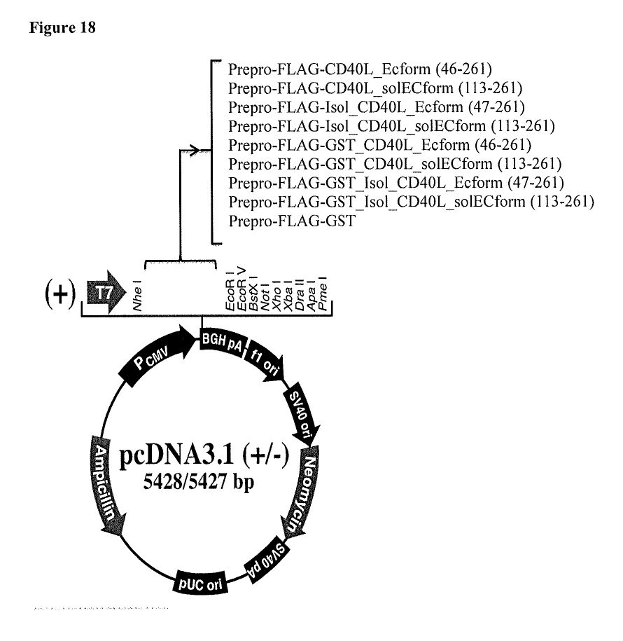

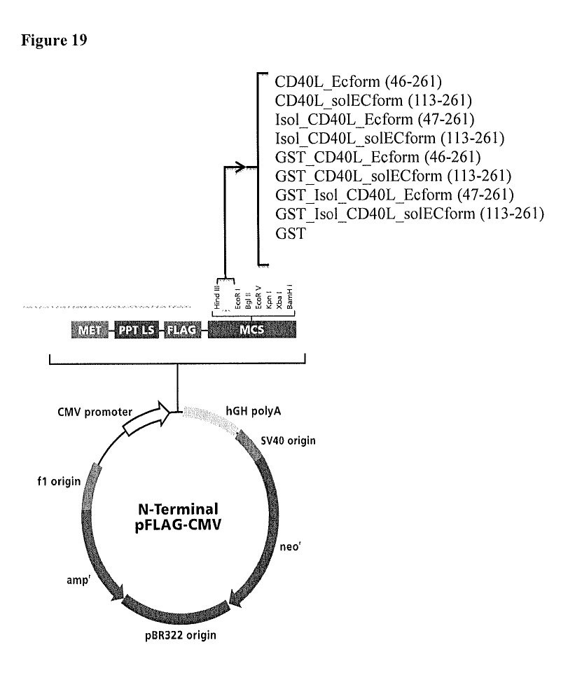

For production of CD40L in mammalian cells and more precisely here in Cos7 cells, two different expression vectors were used: pcDNA3.1 (Invitrogen) and pFLAG-CMV3 (Sigma). For the first one, PCR fragments coding for the different forms of CD40L preceded at the N-terminal part by a sequence coding for a signal peptide for preprotrypsinogen followed by a FLAG sequence have been cloned in pcDNA3.1 (FIG. 18). The second expression vector was produced by cloning the different forms of CD40L into pFLAG which already contains a preprotrypsinogen signal peptide and a FLAG sequence (FIG. 19).

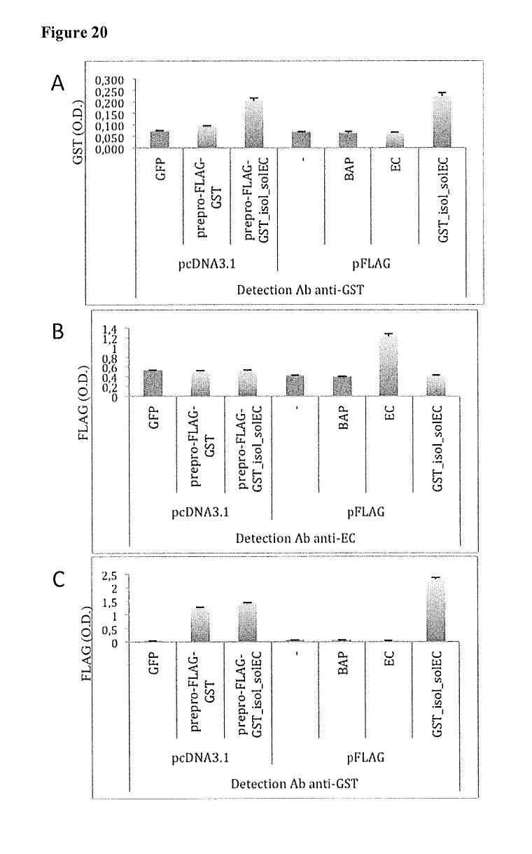

For production, Cos7 cells were transfected with the different constructions made above using Transfectin reagents; and 32 h later, culture medium was recovered in order to check the protein production by ELISA anti-GST and anti-FLAG. The first one allows detection of GST-fusion proteins; and the second one by using two different detection antibodies (i.e. anti-ECform Ab and anti-GST Ab), which allows for detection of the EC form of CD40L uncoupled to GST when using anti-ECform Ab and the detection of all of our GST-fusionned proteins cloned into the chosen eukaryotic expression vectors when using anti-GST Ab. FIG. 20 shows the results of anti-GST ELISA (FIG. 20A), anti-FLAG ELISA using anti-ECform Ab (FIG. 20B), and anti-GST Ab (FIG. 20C). We can see in the FIG. 20A that Cos7 cells have produced significant amount of the GST-fusion soluble extracellular form of CD40L with either pcDNA3.1 or pFLAG when compared with the controls (pcDNA3.1-GFP or empty pFLAG and pFLAG-Bacterial Alkaline Phosphatase (BAP)), and less GST proteins with pcDNA3.1 vector than GST-fusion proteins produced in pcDNA3.1, but significantly more than the control pcDNA3.1-GFP which does not contain GST. FIG. 20B shows that we managed to produce and detect significant amount of the extracellular form of CD40L with the anti-FLAG ELISA using anti-EC form Ab; this form was the only one that could be detected by this antibody. We confirmed the preceding ELISA with the results displayed in FIG. 20C where we detected with an anti-FLAG ELISA and anti-GST detection Ab significant amount of all the GST-fusion forms of CD40L and GST proteins.

### Functionality Test

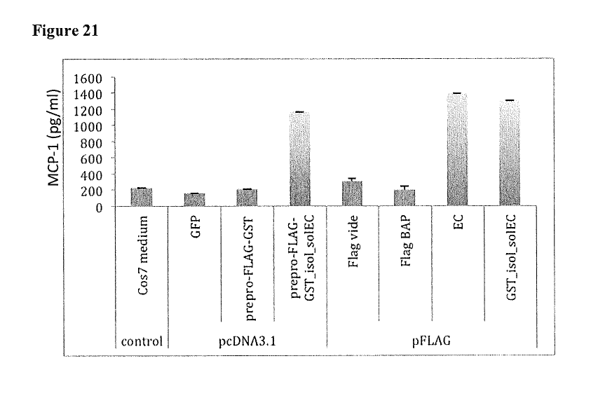

The supernatant of Cos7 cells containing the recombinant proteins were tested in our agonistic test. In this case, after IFN-γ-stimulated overexpression of CD40 receptor at the surface of endothelial cells, the culture medium was removed and replaced by the supernatant of Cos7 cells containing the proteins of interest. 16 hours later, the culture medium was recovered and the production of MCP-1 by endothelial cells was evaluated by ELISA. FIG. 21 shows that the CD40L forms produced by Cos7 cells transfected with the construction performed with the expression vectors pcDNA3.1 or pFLAG were able to induce a significant increase in MCP-1 production by bovine aortic endothelial cells when compared with their relative controls. This shows that the effect is specific of the CD40L proteins produced in the supernatant.

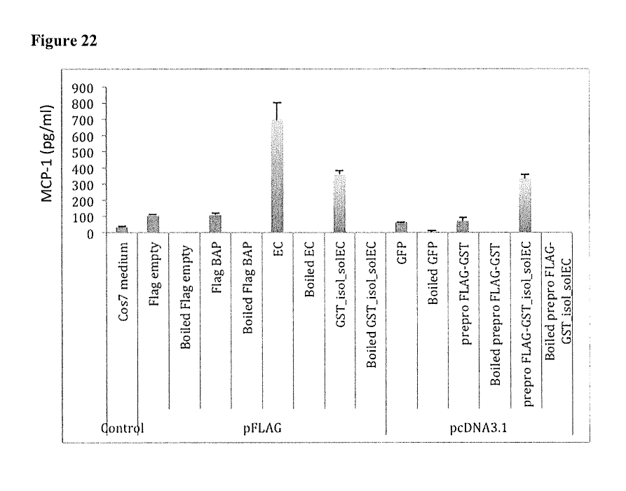

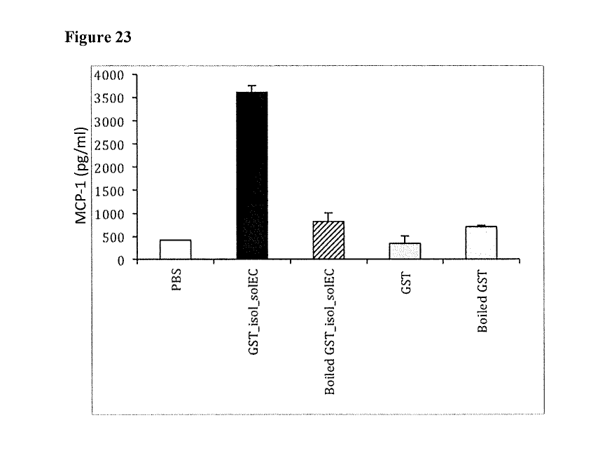

The same test was performed with boiled-Cos7 supernatant in order to determine whether the effect observed in our agonistic test is specifically due to our proteins. FIG. 22 shows that boiling of the supernatant containing the CD40L proteins reduced the production of MCP-1 by endothelial cells.

### Conclusion

Our aim is to develop an efficient vaccine against S. aureus (or other intracellular bacteria)-induced mastitis by targeting the intracellular form of the pathogen by induction of a specific cytotoxic cellular response mediated by CD8+ cells. The proposed strategy is to stimulate the CD40 receptor at the surface of dendritic cells with the CD40L in combination with HKSA. Indeed, the activation of the CD40 signalling pathway in dendritic cells in presence of an antigen leads to induction of a specific cytotoxic response against this antigen. As no bovine CD40L was available, we aimed at producing it. In order to get rapidly sufficient amount of CD40L to work, we began to perform plasmid construction for prokaryotic production which is known to give high protein yields. The objective was to produce proteins that are able to trimerize. Indeed, aggregation of CD40 molecules in trimers following interaction with CD40L is an important initiating step for CD40-mediated signalling. Furthermore, trimerization of CD40L is a prerequisite for the subsequent CD40 multimerization in order to initiate the intracellular signalling cascade. So we have performed constructions coding for the total extracellular domain of CD40L or a shorter form of this extracellular domain preceded, or not, by an isoleucine zipper to encourage trimerization. The first attempts were not successful as the produced proteins were totally insoluble. After testing a lot of protein extraction protocols, we decided to produce our CD40L forms fused to GST for two reasons: 1) to facilitate the protein purification; and 2) to determine whether GST is able to increase the solubility of our proteins. With these constructions, we managed to get our proteins in a non-denaturating buffer. After purification and detoxification on specific columns, we first did not observe the specific effect of the produced proteins in our agonistic test, as the LPS pollution was prevalent. After several tested protocols, we finally managed to get recombinant proteins free of LPS contamination by adding Triton x-100 to the buffers, except in the last wash buffer and the elution buffer. These proteins were functional and induced the activation of the CD40 pathway as observed in our agonistic test. These detoxified proteins can be used in combination with HKSA for cattle vaccination against subsequent S. aureus-induced mastitis.

At the same time, we developed constructions in expression vectors usable in eukaryotic mammalian cells. We modified the construction by adding different signal peptides or by adding GST to some forms. We also developed two different ELISAs to more sensitively detect the proteins in the medium. We then managed to produce and detect soluble CD40L in the medium of Cos7 cells transfected with the constructions. These proteins were tested in the agonistic test and were found to be functional and exempt of LPS as produced in sterile condition.

In conclusion, we managed to produce recombinant CD40L usable as a vaccine adjuvant. Moreover we have managed to produce this protein in expression plasmids usable in mammalian cells. These plasmids could thus be used for DNA vaccination, i.e. a vaccine containing a vector coding for the proteins, here CD40L proteins which serves as adjuvant, combined with an antigen, e.g. HKSA. The advantage of DNA vaccination is the low cost necessary to produce the vectors included in the vaccine. Moreover, the proteins are not produced recombinantly, but by the cells of host animal receiving the vaccine which leads to amplification of the immune response.

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

### LITERATURE REFERENCES

[1] Quezada S, Jarvinen L, Lind E, Noelle R. CD40/CD154 interactions at the interface of tolerance and immunity. Annu Rev Immunol 2004; 22:307-28.
[2] Middleton J R. Staphylococcus aureus antigens and challenges in vaccine development. Expert Rev Vaccines 2008 August; 7(6):805-15.
[3] Hebert A, Sayasith K, Senechal S, Dubreuil P, Lagace J. Demonstration of intracellular Staphylococcus aureus in bovine mastitis alveolar cells and macrophages isolated from naturally infected cow milk. FEMS Microbiol Lett 2000 Dec. 1; 193(1):57-62.
[4] Brouillette E, Grondin G, Shkreta L, Lacasse P, Talbot B G. In vivo and in vitro demonstration that Staphylococcus aureus is an intracellular pathogen in the presence or absence of fibronectin-binding proteins. Microb Pathog 2003 October; 35(4):159-68.
[5] Schoenberger S P, Toes R E, van der Voort E I, Offring a R, Melief C J. T-cell help for cytotoxic T lymphocytes is mediated by CD40-CD40L interactions. Nature 1998 Jun. 4; 393(6684):480-3.
[6] Rolph M S, Kaufmann S H. CD40 signaling converts a minimally immunogenic antigen into a potent vaccine against the intracellular pathogen Listeria monocytogenes. J Immunol 2001 Apr. 15; 166(8):5115-21.
[7] Wells J, Cowled C, Farzaneh F, Noble A. Combined triggering of dendritic cell receptors results in synergistic activation and potent cytotoxic immunity. J Immunol 2008 September; 181(5):3422-31.
[8] Aichele P, Brduscha-Riem K, Oehen S, Odermatt B, Zinkernagel R M, Hengartner H, Pircher H. Peptide antigen treatment of naive and virus-immune mice: antigen-specific tolerance versus immunopathology. Immunity. 1997 May; 6(5):519-29.
[9] Lim S Y, Bauermeister A, Kjonaas R A, Ghosh S K. Phytol-based novel adjuvants in vaccine formulation: 2. Assessment of efficacy in the induction of protective immune responses to lethal bacterial infections in mice. J Immune Based Ther Vaccines. 2006 Oct. 23; 4:5.

What is claimed is:

1. A combination for treating and/or preventing of mastitis, said combination comprising
i) an agonistic anti-CD40 monoclonal antibody or a CD40 ligand or a vector containing nucleic acids encoding the anti-CD40 monoclonal antibody or a vector containing a nucleic acids encoding the CD40 ligand; and
ii) inactivated or attenuated bacteria selected from the group consisting of Staphylococcus, Streptococcus, Listeria or Escherichia for use in the treatment and/or prevention of mastitis.

2. The combination of claim 1, wherein the agonistic anti-CD40 monoclonal antibody comprises a) an immunoglobulin heavy chain variable domain (VH) which comprises the hypervariable regions CDR1, CDR2 and CDR3, said CDR1 having the amino acid sequence of SEQ ID NO:1, said CDR2 having the amino acid sequence of SEQ ID NO:2, and said CDR3 having the amino acid sequence of SEQ ID NO:3; and b) an immunoglobulin light chain variable domain (VL) which comprises the hypervariable regions CDR1′, CDR2′ and CDR3′, said CDR1′ having the amino acid sequence of SEQ ID NO:4, said CDR2′ having the amino acid sequence of SEQ ID NO:5, and said CDR3′ having the amino acid sequence of SEQ ID NO:6.

3. The combination of claim 1, wherein the agonistic anti-CD40 monoclonal antibody is an anti-bovine CD40 antibody.

4. The combination of claim 1, wherein the anti-CD40 monoclonal antibody is produced by the hybridoma deposited at Belgian Coordinated Collections of Microorganisms under No: LMBP 7218CB.

5. The combination of claim 1, wherein the Staphylococcus is Staphylococcus aureus or Staphylococcus agalactiae, the Streptococcus is Streptococcus uberis, the Escherichia is Escherichia coli, and the Listeria is Listeria monocytogenes.

6. The combination of claim 1, wherein component i) is sufficient to induce CD40 receptor aggregation.

7. The combination of claim 1, wherein the CD40 ligand is soluble.

8. The combination of claim 1, wherein the CD40 ligand comprises amino acids 47 to 261 or 46 to 261 of SEQ ID NO:26 or SEQ ID NO:27, or a biological active fragment thereof.

9. The combination of claim 1, wherein the CD40 ligand is a fusion protein with glutathione S-transferase (GST).

10. A nucleic acid molecule encoding an agonistic anti-CD40 monoclonal antibody.

11. The nucleic acid molecule of claim 10, wherein the agonistic anti-CD40 monoclonal antibody comprises a) an immunoglobulin heavy chain variable domain (VH) which comprises the hypervariable regions CDR1, CDR2 and CDR3, said CDR1 having the amino acid sequence of SEQ ID NO:1, said CDR2 having the amino acid sequence of SEQ ID NO:2, and said CDR3 having the amino acid sequence of SEQ ID NO:3; and b) an immunoglobulin light chain variable domain (VL) which comprises the hypervariable regions CDR1′, CDR2′ and CDR3′, said CDR1′ having the amino acid sequence of SEQ ID NO:4, said CDR2′ having the amino acid sequence of SEQ ID NO:5, and said CDR3′ having the amino acid sequence of SEQ ID NO:6.

12. A vector comprising the nucleic acid molecule according to claim 10.

13. A host cell comprising the vector according to claim 12.

14. A nucleic acid molecule encoding a CD40 ligand.

15. The nucleic acid molecule of claim 14, wherein the CD40 ligand comprises amino acids 47 to 261 or 46 to 261 of SEQ ID NO:26 or SEQ ID NO:27, or a biological active fragment thereof.

16. A vector comprising the nucleic acid molecule of claim 14.

17. A host cell comprising the vector of claim 16.

18. A vaccine comprising a combination containing
i) an agonistic anti-CD40 monoclonal antibody or a CD40 ligand or a vector containing nucleic acids encoding the anti-CD40 monoclonal antibody or a vector containing a nucleic acids encoding the CD40 ligand; and
ii) inactivated or attenuated bacteria selected from the group consisting of Staphylococcus, Streptococcus, Listeria or Escherichia for use in the treatment and/or prevention of mastitis.

19. The vaccine of claim 18, wherein the vaccine is to be administered subcutaneously, intramammary or intramuscular.

20. The vaccine of claim 18, for use in dairy cattle, sheep or goats.
